(19) United States
(12) Patent Application Publication
(10) Pub. No.: US 2021/0372959 A1
(43) Pub. Date: Dec. 2, 2021

(54) Nanopore Method for Identifying Single Amino Acid in Oligopeptides

(71) Applicant: University of South Carolina, Columbia, SC (US)

(72) Inventors: Chang Liu, Chapin, SC (US); Qian Wang, Colurmbia, SC (US)

(73) Assignee: University of South Carolina, Columbia, SC (US)

(21) Appl. No.: 17/212,021

(22) Filed: Mar. 25, 2021

Related U.S. Application Data
(60) Provisional application No. 63/029,764, filed on May 26, 2020.

(51) Int. Cl. G01N 27/12; G01N 33/68

(52) U.S. Cl. CPC G01N 27/128; G01N 33/6806

(57) ABSTRACT

The current disclosure provides a transformative concept based on nanopore technology, Sequencing-by-Hydrolysis, to identify the N-terminal amino acid and the length of each peptide fragment in a peptide ladder to reconstitute the sequence of a protein: a protein/peptide analyte will be nonspecifically hydrolyzed to generate random fragments of the analyte that are different by one amino acid with the N-terminal amino acid of each fragment modified so it generates a distinguishable fingerprint signal when tested by nanopore. The length of the fragment can be estimated by characterizing its translocation signal to back calculate the location of the amino acid in the original analyte. This approach will significantly advance the nanopore technology with single amino acid resolution for protein/peptide sequencing.

### STATEMENT REGARDING FEDERALLY SPONSORED RESEARCH

This disclosure was made with government support under K22 AI136686 by the National Institute of Health. The government may have certain rights in the invention.

### TECHNICAL FIELD

The present invention relates to a transformative concept based on nanopore technology, Sequencing-by-Hydrolysis, to identify the N-terminal amino acid and the length of each peptide fragment in a peptide ladder to reconstitute the sequence of a protein.

### BACKGROUND

Nanopores in biological and synthetic membranes have been developed to detect and characterize a variety of analytes at the single-molecule level. While nanopores have shown great promise for sequencing of nucleic acid molecules, there is much more enthusiasm surrounding the idea of applying the nanopore technology to sequence proteins/peptides. However, existing studies only achieved identification of peptide or quadromers in its entirety.

Accordingly, it is an object of the present invention to overcome the above failings. The current disclosure provides a transformative concept based on nanopore technology, Sequencing-by-Hydrolysis, to identify the N-terminal amino acid and the length of each peptide fragment in a peptide ladder to reconstitute the sequence of a protein. Specifically, a protein/peptide analyte will be nonspecifically hydrolyzed to generate random fragments of the analyte that are different by one amino acid (ladder). The N-terminal amino acid of each fragment will be modified so it generates a distinguishable fingerprint signal when tested by nanopore. The length of the fragment can be estimated by characterizing its translocation signal to back calculate the location of the amino acid in the original analyte. This approach will significantly advance the nanopore technology with single amino acid resolution for protein/peptide sequencing.

Citation or identification of any document in this application is not an admission that such a document is available as prior art to the present disclosure.

### SUMMARY

The above objectives are accomplished according to the present disclosure by providing in a first embodiment, a method for identifying individual amino acids. The method may include employing a biosensing strategy using at least one nanopore, N-terminal derivatization of at least one amino acid to form an amino acid analyte, and differentiating individual amino acid analytes from one another via analysis of the at least one analyte interacting with the at least one nanopore. Further, the method may include developing a characteristic profile for each individual amino acid via a statistical description of each individual amino acid analyte's translocation process through the at least one nanopore. Still, the method may include analyzing blockade and dwell times for the individual amino acid analytes within the at least one nanopore. Yet again, the nanopore may be an α-hemolysin nanopore. Further again, the method may include employing an aromatic tag as part of the N-terminal derivatization. Moreover, N-terminal derivatization may employ derivatization reagents such as 2,3-naphthalenedicarboxaldehyde (NDA) and/or 2-naphthylisothiocyanate (NITC). Again yet, identifying at least one individual amino acid may be accomplished via analyzing current blockade induced via presence of the at least one amino acid analyte. Further still, identifying at least one individual amino acid may be accomplished via analyzing dwell time induced via the at least one amino acid analyte when analyzing current blockage is ineffective at identifying the at least one amino acid. Yet further, the method may include generating a signal on an electrical current trace characterized by current blockade and dwell time when the at least one individual amino acid analyte translocates the at least one nanopore.

In a further embodiment, a method for identifying individual amino acids is provided. The method may include inserting at least one nanopore into a phosphate lipid bilayer and the phosphate lipid bilayer separates cis and trans compartments in an electrolyte solution, applying an external positive voltage to the trans facing side of the bilayer, grounding the cis facing side of the bilayer, determining amino acid analyte insertion via an absolute value of open pore current under positive and negative voltages, and identifying at least one individual amino acid via interaction of an amino acid analyte with the at least one nanopore. Further, the method may include the tail of the at least one nanopore inserted into the phosphate lipid bilayer with the head of the at least one nanopore remaining in the cis compartment. Still yet, the at least one nanopore may comprise α-hemolysin nanopore. Again, the method may include introducing a sample of at least one individual amino acid analyte to the cis compartment. Moreover, introduction of at least one amino acid derivative in the cis compartment may induce transient events in an ionic current flowing through the at least one nanopore. Further yet, the method may characterize capture of at least one amino acid analyte via analysis of current blockade and blockade duration within the at least one nanopore. Still, identifying at least one individual amino acid may be accomplished via analyzing current blockade induced via presence of the at least one individual amino acid analyte. Furthermore, identifying at least one individual amino acid may be accomplished via analyzing dwell time induced via presence of the at least one individual amino acid analyte when analyzing current blockage is ineffective at identifying the at least one amino acid analyte. Still further, the method may generate a signal on an electrical current trace characterized by current blockade and dwell time when the at least one individual amino acid analyte translocates the at least one nanopore.

These and other aspects, objects, features, and advantages of the example embodiments will become apparent to those having ordinary skill in the art upon consideration of the following detailed description of example embodiments.

### BRIEF DESCRIPTION OF THE DRAWINGS

An understanding of the features and advantages of the present disclosure will be obtained by reference to the following detailed description that sets forth illustrative embodiments, in which the principles of the disclosure may be utilized, and the accompanying drawings of which:

FIG. 1 shows at (a) a schematic illustration of the identification strategy of single amino acid derivatives through an α-HL nanopore, at (b) synthetic routes of different amino acid derivatives, and at (c) translocation events frequency of raw materials and various derivatives through α-HL nanopores at the same experimental condition.

FIG. 2 shows at (a) and (b) molecular structures and corresponding contour plots depicting the blockade (I/I₀) vs. dwell time distribution at the same applied potential for different AA derivatives: (a) PITC-derivatives, (b) OPA-derivatives, and at (c) and (d) translocation event frequency distribution of blockade (I/I₀) and dwell time for (c) PITC and (d) OPA derivatives.

FIG. 3 shows at (a) and (b) from top to bottom: molecular structure, representative current trace of translocations, and corresponding contour plots of different (a) NITC-derivatives and (b) NDA-derivatives, and at (c) and (d) translocation event frequency distribution of blockade (I/I₀) and dwell time for (c) NITC-derivatives and (d) NDA-derivatives.

FIG. 4 shows discrimination of each amino acid in a mixture sample.

FIG. 5 shows Table 1, discriminant analysis of five NITC-AAs using Mahalanobi's distance matrices.

FIG. 6 shows Table 2, discriminant analysis of five NITC-AAs using histogram binning method.

FIG. 7 shows at (a) change of open pore current value when changing voltage (100 mV) direction after one α-HL nanopore is inserted into the lipid membrane, at (b) current trace of the lipid membrane with capacitance of 160 pF during the stirring process, and at (c) current trace of translocations observed by one α-HL nanopore on the lipid membrane after adding the sample and stirring for a few seconds.

FIG. 8 shows representative current trace of translocations of the raw materials through α-HL nanopores (final concentration 200 μM) at (a) amino acids, and at (b) derivatization reagents.

FIG. 9 shows space-filling structures of five selected amino acids based on molecular modeling with Q-chem 4.3 software package.

FIG. 10 shows space-filling structures of (a) PITC derivatized AAs and (b) OPA derivatized AAs calculated using the Q-chem 4.3 software package.

FIG. 11 shows at (a), from top to bottom: molecular structure and corresponding contour plots of different NITC-derivatives in repeated experiments, and at (b) and (c) translocation event frequency distributions of current blockade (b) and dwell time (c) of each NITC-derivative, respectively.

FIG. 12 shows space-filling structures of (a) NITC derivatized AAs and (b) NDA derivatized AAs calculated using the Q-chem 4.3 software package.

FIG. 13 shows Table S1, structures of different amino acid derivatives.

FIG. 14 shows HPLC spectrum of PITC-Tyr.

FIG. 15 shows HPLC spectrum of PITC-Phe.

FIG. 16 shows HPLC spectrum of PITC-Ala.

FIG. 17 shows HPLC spectrum of PITC-Asp.

FIG. 18 shows HPLC spectrum of PITC-His.

FIG. 19 shows HPLC spectrum of NITC-Tyr.

FIG. 20 shows HPLC spectrum of NITC-Phe.

FIG. 21 shows HPLC spectrum of NITC-Ala.

FIG. 22 shows HPLC spectrum of NITC-Asp.

FIG. 23 shows HPLC spectrum of NITC-His.

FIG. 24 shows HPLC spectrum of OPA-Tyr.

FIG. 25 shows HPLC spectrum of OPA-Phe.

FIG. 26 shows HPLC spectrum of OPA-Ala.

FIG. 27 shows HPLC spectrum of OPA-Asp.

FIG. 28 shows HPLC spectrum of OPA-His.

FIG. 29 shows HPLC spectrum of NDA-Tyr.

FIG. 30 shows HPLC spectrum of NDA-Phe.

FIG. 31 shows HPLC spectrum of NDA-Ala.

FIG. 32 shows HPLC spectrum of NDA-Asp.

FIG. 33 shows HPLC spectrum of NDA-His.

FIG. 34 shows ¹H NMR spectrum of PITC-Tyr.

FIG. 35 shows ¹³C NMR spectrum of PITC-Tyr.

FIG. 36 shows ¹H NMR spectrum of PITC-Phe.

FIG. 37 shows ¹³C NMR spectrum of PITC-Phe.

FIG. 38 shows ¹H NMR spectrum of PITC-Ala.

FIG. 39 shows ¹³C NMR spectrum of PITC-Ala.

FIG. 40 shows ¹H NMR spectrum of PITC-Asp.

FIG. 41 shows ¹³C NMR spectrum of PITC-Asp.

FIG. 42 shows ¹H NMR spectrum of PITC-His.

FIG. 43 shows ¹³C NMR spectrum of PITC-His.

FIG. 44 shows ¹H NMR spectrum of NITC-Tyr.

FIG. 45 shows ¹³C NMR spectrum of NITC-Tyr.

FIG. 46 shows ¹H NMR spectrum of NITC-Phe.

FIG. 47 shows ¹³C NMR spectrum of NITC-Phe.

FIG. 48 shows ¹H NMR spectrum of NITC-Ala.

FIG. 49 shows ¹³C NMR spectrum of NITC-Ala.

FIG. 50 shows ¹H NMR spectrum of NITC-Asp.

FIG. 51 shows ¹³C NMR spectrum of NITC-Asp.

FIG. 52 shows ¹H NMR spectrum of NITC-His.

FIG. 53 shows ¹³C NMR spectrum of NITC-His.

FIG. 54 shows ¹H NMR spectrum of OPA-Tyr.

FIG. 55 shows ¹³C NMR spectrum of OPA-Tyr.

FIG. 56 shows ¹H NMR spectrum of OPA-Phe.

FIG. 57 shows ¹³C NMR spectrum of OPA-Phe.

FIG. 58 shows ¹H NMR spectrum of OPA-Ala.

FIG. 59 shows ¹³C NMR spectrum of OPA-Ala.

FIG. 60 shows ¹H NMR spectrum of OPA-Asp.

FIG. 61 shows ¹³C NMR spectrum of OPA-Asp.

FIG. 62 shows ¹H NMR spectrum of OPA-His.

FIG. 63 shows ¹³C NMR spectrum of OPA-His.

FIG. 64 shows ¹H NMR spectrum of NDA-Tyr.

FIG. 65 shows ¹³C NMR spectrum of NDA-Tyr.

FIG. 66 shows ¹H NMR spectrum of NDA-Phe.

FIG. 67 shows ¹³C NMR spectrum of NDA-Phe.

FIG. 68 shows ¹H NMR spectrum of NDA-Ala.

FIG. 69 shows ¹³C NMR spectrum of NDA-Ala.

FIG. 70 shows ¹H NMR spectrum of NDA-Asp.

FIG. 71 shows ¹³C NMR spectrum of NDA-Asp.

FIG. 72 shows ¹H NMR spectrum of NDA-His.

FIG. 73 shows ¹³C NMR spectrum of NDA-His.

FIG. 74 shows MS spectrum of PITC-Tyr.

FIG. 75 shows MS spectrum of PITC-Phe.

FIG. 76 shows MS spectrum of PITC-Asp.

FIG. 77 shows MS spectrum of PITC-His.

FIG. 78 shows MS spectrum of NITC-His.

FIG. 79 shows MS spectrum of OPA-Tyr.

FIG. 80 shows MS spectrum of OPA-Phe.

FIG. 81 shows MS spectrum of OPA-Ala.

FIG. 82 shows MS spectrum of OPA-Asp.

FIG. 83 shows MS spectrum of OPA-His.

FIG. 84 shows MS spectrum of NDA-Tyr.

FIG. 85 shows MS spectrum of NDA-Phe.

FIG. 86 shows MS spectrum of NDA-Ala.

FIG. 87 shows MS spectrum of NDA-Asp.

FIG. 88 shows MS spectrum of NDA-His.

FIG. 89 shows at: (a) schematic of NDA and NITC derivatization of 9 amino acids in 3 classes and the experimental setup (not to scale); (b) representative fragment of ionic current recording before (open pore) and after (translocation events) NITC-Tyr derivatives were added; (c) an illustration of typical signal events caused by translocation blockade; and (d) histogram of events per bin of current blockade I/I₀ (left versus bottom axes) and scatter-plot of dwell time versus I/I₀ (right versus bottom axes) produced by NITC-Tyr in an α-HL nanopore.

FIG. 90 shows superimposed histograms of I/I₀ obtained from nanopores for NDA-modified amino acids, analyzed individually and grouped as: (a) polar, (b) charged, (c) non-polar amino acids with superimposed histograms of dwell time for the derivatives; and (d) mean relative current blockade and standard deviation produced by each NDA amino acid derivative versus its spatial volume.

FIG. 91 shows superimposed histograms of I/I₀ obtained from the nanopore for NITC-modified amino acids, analyzed individually and grouped as: (a) polar, (b) charged, (c) non-polar amino acids; and (d) mean relative current blockade and standard deviation produced by each NITC amino acid derivative versus its spatial volume.

The figures herein are for illustrative purposes only and are not necessarily drawn to scale.

### DETAILED DESCRIPTION OF A PREFERRED EMBODIMENT

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting.

Unless specifically stated, terms and phrases used in this document, and variations thereof, unless otherwise expressly stated, should be construed as open ended as opposed to limiting. Likewise, a group of items linked with the conjunction “and” should not be read as requiring that each and every one of those items be present in the grouping, but rather should be read as “and/or” unless expressly stated otherwise. Similarly, a group of items linked with the conjunction “or” should not be read as requiring mutual exclusivity among that group, but rather should also be read as “and/or” unless expressly stated otherwise.

Furthermore, although items, elements or components of the disclosure may be described or claimed in the singular, the plural is contemplated to be within the scope thereof unless limitation to the singular is explicitly stated. The presence of broadening words and phrases such as “one or more,” “at least,” “but not limited to” or other like phrases in some instances shall not be read to mean that the narrower case is intended or required in instances where such broadening phrases may be absent.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are cited to disclose and describe the methods and/or materials in connection with which the publications are cited. All such publications and patents are herein incorporated by references as if each individual publication or patent were specifically and individually indicated to be incorporated by reference. Such incorporation by reference is expressly limited to the methods and/or materials described in the cited publications and patents and does not extend to any lexicographical definitions from the cited publications and patents. Any lexicographical definition in the publications and patents cited that is not also expressly repeated in the instant application should not be treated as such and should not be read as defining any terms appearing in the accompanying claims. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Where a range is expressed, a further embodiment includes from the one particular value and/or to the other particular value. The recitation of numerical ranges by endpoints includes all numbers and fractions subsumed within the respective ranges, as well as the recited endpoints. Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure. For example, where the stated range includes one or both of the limits, ranges excluding either or both of those included limits are also included in the disclosure, e.g. the phrase “x to y” includes the range from ‘x’ to ‘y’ as well as the range greater than ‘x’ and less than ‘y’. The range can also be expressed as an upper limit, e.g. ‘about x, y, z, or less’ and should be interpreted to include the specific ranges of ‘about x’, ‘about y’, and ‘about z’ as well as the ranges of ‘less than x’, less than y’, and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than y’, and ‘greater than z’. In addition, the phrase “about ‘x’ to ‘y’”, where ‘x’ and ‘y’ are numerical values, includes “about ‘x’ to about ‘y’”.

It should be noted that ratios, concentrations, amounts, and other numerical data can be expressed herein in a range format. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about,” it will be understood that the particular value forms a further aspect. For example, if the value “about 10” is disclosed, then “10” is also disclosed.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

As used herein, the singular forms “a”, “an”, and “the” include both singular and plural referents unless the context clearly dictates otherwise.

As used herein, “about,” “approximately,” “substantially,” and the like, when used in connection with a measurable variable such as a parameter, an amount, a temporal duration, and the like, are meant to encompass variations of and from the specified value including those within experimental error (which can be determined by e.g. given data set, art accepted standard, and/or with e.g. a given confidence interval (e.g. 90%, 95%, or more confidence interval from the mean), such as variations of +/−10% or less, +/−5% or less, +/−1% or less, and +/−0.1% or less of and from the specified value, insofar such variations are appropriate to perform in the disclosure. As used herein, the terms “about,” “approximate,” “at or about,” and “substantially” can mean that the amount or value in question can be the exact value or a value that provides equivalent results or effects as recited in the claims or taught herein. That is, it is understood that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

The term “optional” or “optionally” means that the subsequent described event, circumstance or substituent may or may not occur, and that the description includes instances where the event or circumstance occurs and instances where it does not.

As used interchangeably herein, the terms “sufficient” and “effective,” can refer to an amount (e.g. mass, volume, dosage, concentration, and/or time period) needed to achieve one or more desired and/or stated result(s). For example, a therapeutically effective amount refers to an amount needed to achieve one or more therapeutic effects.

Various embodiments are described hereinafter. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s). Reference throughout this specification to “one embodiment”, “an embodiment,” “an example embodiment,” means that a particular feature, structure or characteristic described in connection with the embodiment is included in at least one embodiment of the present disclosure. Thus, appearances of the phrases “in one embodiment,” “in an embodiment,” or “an example embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the disclosure. For example, in the appended claims, any of the claimed embodiments can be used in any combination.

All patents, patent applications, published applications, and publications, databases, websites and other published materials cited herein are hereby incorporated by reference to the same extent as though each individual publication, published patent document, or patent application was specifically and individually indicated as being incorporated by reference.

Nanopore technology is a promising alternative proteomic tool for protein sequencing in point-of-care and resource-limited settings. Existing nanopore studies have only achieved identification of oligopeptides in their entirety due to the lack of single amino acid distinguishability. The current disclosure provides a Sequencing-by-Hydrolysis (SBH) method to develop a nanopore-based protein/peptide sequencer with single amino acid resolution towards de novo sequencing.

The primary sequence of a protein or a peptide is essential to its identification and function. In the area of personalized diagnosis and therapeutics, accurate proteomic information of proteins or peptides as biomarkers can much better reflect an individual's health status than the genomic information. Classical proteomics techniques, such as mass spectrometry (MS), are less sensitive and reproducible when detecting low abundance proteins/peptides, and are also time consuming and too expensive for medical diagnostics. Nanopore technology is a promising alternative because of its single-molecule analysis capacity and simplicity.

However, while the technology is maturing in DNA detection and sequencing, it still cannot sequence protein/peptide due to the lack of single amino acid distinguishability. To address this issue, the current disclosures provides an innovative Sequencing-by-Hydrolysis (SBH) nanopore-based method to identify the N-terminal amino acid of each peptide fragment in a peptide ladder generated from a peptide analyte to reconstitute its full-length sequence. The project will focus on the proof-of-concept of identifying the N-terminal amino acid and the length of different oligopeptides as the first step towards de novo protein/peptide sequencing in clinical, point-of-care, and resource-limited settings by nanopore sequencers.

This approach has several unique features that distinguish it from other known research efforts. First, a crucial point for nanopore sensing is the effective diameter and length of the sensing region (i.e. the constriction region). In existing nanopore technologies, several amino acid residues usually engage the sensing region of the pore at the same time, leading to a joint effect to the measurement, which prevents single amino acid resolution. Instead of trying to resolute each amino acid on a peptide sequentially, we propose to identify only the N-terminal amino acid on every hydrolyzed fragment from a peptide ladder at first, and then reconstitute the sequence of the peptide. The current disclosure functionalizes (derivatize) the N-terminal end of each oligopeptide with an optimized tag to generate distinguishable fingerprint signals. Second, efficient conjugation chemistry will be employed to in situ derivatize the N-terminal end of amino acids or peptides to control the net charge distribution, ensure the anisotropic structural feature and prolong the interaction with the lumen face of the nanopore. Third, the nanopore biosensor detects each peptide fragment translocation at single molecular level to identify its N-terminal amino acid and length by analyzing signal amplitude, dwell time, and roughness, etc. using automated algorithms. The output results can be readily input to bioinformatic analysis for sequencing. Fourth, the current disclosure provides nanopore devices that may be fabricated with precision and reproducibility suitable for clinical applications. The use of a hemolysin-based nanopore allows the adoption of a large variety of well-established protocols to ensure fabrication quality. These processes produce highly consistent devices to ensure reproducible results.

Nanopore technology has been employed as a powerful tool for DNA sequencing and analysis. To extend this method to peptide sequencing, a necessary step is to profile individual amino acids (AAs) through their nanopore stochastic signals, which remains a great challenge due to the low signal-to-noise ratio and unpredictable conformational changes of AAs during their translocation through nanopores. We showed that the combination of an N-terminal derivatization strategy of AAs with nanopore technology could lead to effective in situ differentiation of AAs. Four different derivatization reactions have been tested with five selected AAs, i.e. Ala, Phe, Tyr, His and Asp. Using an α-Hemolysin (α-HL) nanopore, we demonstrated the feasibility of derivatization-assisted identification of AAs regardless of their charge composition and polarity. The method was further applied to discriminate each individual AA in testing datasets using their established nanopore profiles from training datasets. We envision this proof-of-concept study will not only pave a way for identification of individual AAs but also lead to future applications in protein/peptide sequencing using the nanopore technology.

Emerging resistive pulse nanopore sensing technology, ranging from biological protein to artificial solid-state nanometer-scale pores, makes it possible to detect, analyze, manipulate and characterize a variety of analytes at the single-molecule level. In general, nanopore sensing operates on a basic structure with a thin membrane containing a single nanopore that separates an ionic solution into two compartments. A transmembrane bias is applied to capture and transport analytes from one side of the membrane to the other through the nanopore. The entry of a molecule into a nanopore could cause a reduction in the latter's ionic conductance. The resulting ionic current blockade depth and the residence time have been shown to provide detailed information on the size, adsorbed charge, and other properties of the molecule. Consequently, nanopore based nucleic acid sequencing technology has been successfully commercialized with single base resolution, label-free detection, and long-read capability. However, to extend this method to peptide sequencing, a large hurdle is to differentiate individual amino acids (AAs) through their nanopore stochastic signals due to the low signal-to-noise ratio and unpredictable conformational changes of AAs during their translocation through nanopores.

To mitigate these challenges, different measurement methods on various nanopores have been developed in attempt to achieve higher sensitivity. In some pioneer studies, the identification of some specific AAs or short peptides by monitoring the ion translocation in perpendicular nanochannel, in recognition tunneling, and in sputtered sub-nanometer pores made on solid-state materials were first demonstrated. Later on, identification of peptides with different lengths by one AA was achieved with wild-type aerolysin nanopore, α-Hemolysin (α-HL) nanopore and viral DNA packaging motor, whereas recognition of proteins and peptides with minor sequence differences was accomplished using FraC nanopores.

Despite the solid foundation laid by these investigations, identification of AAs by nanopore technology is still limited by the lack of characteristics in the interaction with nanopores because of the much smaller size and the fast translocation rate of the AAs. To utilize the robust structure of biological nanopores, alternative methods such as decreasing the diameter of the pore lumen, or increasing the volume of AAs by efficient and versatile chemical modifications were used to achieve more AA-pore interactions during translocation. A simulation study proposes that nanoporous single-layer MoS₂ can detect individual AAs in a polypeptide chain, but the results have not been experimentally proven. An elegant design by Bayley and others incorporated the usage of metal-organic complexes into the biological nanopore, which could effectively differentiate amino acid enantiomers. Recently, aerolysin with a narrow constriction of ˜1.0 nm was favored as a biological nanopore for recognizing AAs due to its highly charged sensing interface. Lu et al. found that the cyclization of cysteine and homocysteine into thiazolidines could enhance the signal differentiation through an aerolysin nanopore. Ying et al. first reported the detection of a single cysteine molecule using the interaction between the aerolysin sensing interface and the analyte. An encouraging study by Oukhaled et al. reported identification of all proteinogenic AAs using an aerolysin nanopore with the help of a short peptide carrier. However, translation of this method to practical protein sequencing seems overwhelmingly challenging.

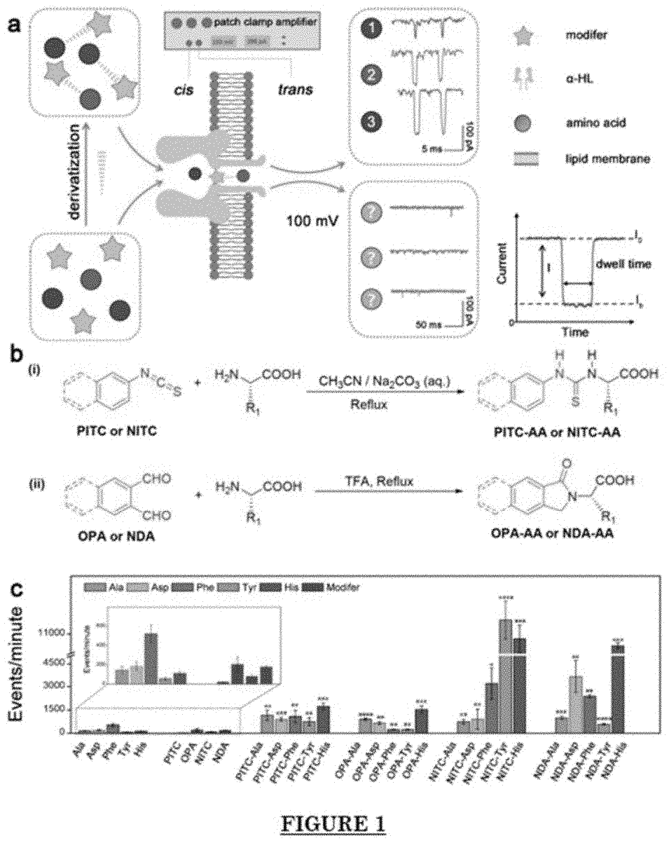

The current disclosure envisions an efficient universal conjugation strategy of AAs could augment the interaction of AA derivatives with the pore lumen surface, and may be readily applicable towards sequencing. Considering the size mismatch between AAs and the α-HL nanopore, N-terminal conjugation can increase the aspect ratio of AAs, leading to a prolonged interaction with the nanopore. Meanwhile, most positively charged AA or peptides can be neutralize by N-terminal conjugation to form more negatively charged final conjugates. Moreover, for future applications in nanopore protein sequencing, the quantitative nature of the N-terminal conjugation method with similar reactivity towards different amino acids and peptides is desired to avoid introducing interfering impurities and any further purification. Among various N-terminal derivatization methods of AAs, in situ ortho-phthalaldehyde (OPA) and phenyl isothiocyanate (PITC) derivatizations are two of the most widely studied due to their high reaction rate and efficiency. As shown in FIG. 1, four modifiers including OPA, 2,3-naphthalenedicarboxaldehyde (NDA), PITC, and 2-naphthylisothiocyanate (NITC) were employed in the N-terminal derivatization of 5 representative AAs in this study. The ionic current blockade events of each AA-derivative were characterized using α-HL nanopores and each derivatization method was evaluated for its ability to distinguish AAs via nanopores. Our results show that NDA and NITC derivatizations were able to facilitate the discrimination of 5 AAs individually. This proof-of-concept study revealed the possibility of individual AAs differentiation using α-HL nanopore technology, which paves the way for future investigations in nanopore based peptide sequencing and analysis.

FIG. 1 shows at (a) schematic illustration of the identification strategy of single amino acid derivatives through α-HL nanopore. The characteristic signals 1, 2, and 3 are from NITC-Ala, NITC-His, and NITC-Tyr, respectively, and at (b) synthetic routes of different amino acid derivatives. Route i is for PITC/NITC derivatives; route ii is for OPA/NDA derivatives, and at (c) translocation events frequency of raw materials and various derivatives through α-HL nanopores at the same experimental condition. Statistical significance is shown between derivatives and corresponding single amino acid (*: 0.01<p<0.05; 0.001<p<0.01; 0.0001<p<0.001; ****: p<0.0001).

Material and Methods

Materials.

Derivative reagents o-phthalaldehyde (OPA), phenyl isothiocyanate (PITC), naphthalene isothiocyanate (NITC) and all amino acids were purchased from Sigma-Aldrich and used without further purification. The derivative reagent 2,3-naphthalenedicarboxaldehyde (NDA) was synthesized according to the reference method, see Mallouli, A.; Lepage, Y. CONVENIENT SYNTHESES OF NAPHTHALENE-2,3-DICARBOXALDEHYDES, ANTHRACENE-2,3-DICARBOXALDEHYDES, AND NAPHTHACENE-2,3-DICARBOXALDEHYDES. Synthesis-Stuttgart 1980, 689-689. The KCl working solution was prepared using deionized water from a Milli-Q water purification system (resistivity of 18.2 MΩ/cm, 25° C., Millipore Corporation) and was filtered through 0.02 μm filter before use. α-HL from Staphylococcus aureus (lyophilized powder, Protein ˜60% by Lowry, ≥10,000 units/mg protein) was purchased from Sigma-Alrich.

General Procedure for Preparing of PITC/NITC Derivatives.

PITC/NITC derivatives were synthesized according to the route i as shown in FIG. 1 at (b). AA (1.2 equiv.) was dissolved in the mixture of acetonitrile and 0.1 M Na₂CO₃ solution, followed by a dropwise addition of PITC/NITC (1.0 equiv.). The reaction mixture was stirred and refluxed overnight. The solvent was then removed, and the precipitate was collected as the crude product, which was washed with 1 M HCl and methanol, and dried to afford the target product.

General Procedure for Preparing of OPA/NDA Derivatives.

OPA/NDA derivatives were synthesized according to the route ii as shown in FIG. 1 at (b). AA (1.0 equiv.) and OPA/NDA (1.2 equiv.) were mixed in acetonitrile together with the catalyst trifluoroacetic acid (1.4 equiv.). The reaction mixture was then refluxed for about 3 h before it was cooled down to room temperature. The yellow precipitate was collected and washed with acetonitrile to afford corresponding product.

Characterization of amino acid derivatives.

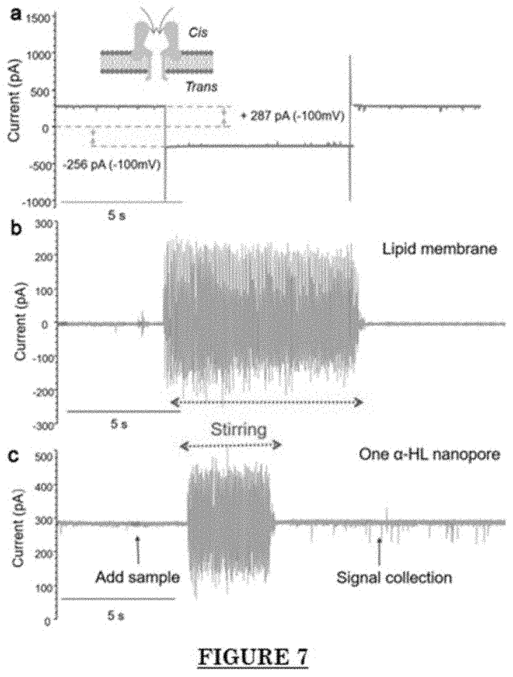

The ¹H and ¹³C NMR spectra were recorded at 298 K in deuterated solvents using Bruker Avance 400 MHz spectrometer. Data are represented as follows: chemical shift, multiplicity (s=singlet, d=doublet, t=triplet, q=quartet, m=multiplet), coupling constants in Hertz (Hz), integration. High-Resolution-Mass-Spectra (HRMS) of amino acid derivative were recorded on Thermo Velos Pro Orbitrap Liquid Chromatography-Mass Spectrometry (LCMS). High performance liquid chromatography (HPLC) of amino acid derivative were recorded on an Agilent 1100 HPLC equipped with a ZORBAX SB-C18 column, see FIG. 7.

Nanopore Fabrication and Low-Noise Electrical Recording.

All electrophysiology experiments were performed on the Planar Lipid Bilayer workstation (Warner Instruments) at room temperature (˜23° C.). Fabrication of α-HL nanopore devices follows traditional method previously reported. Briefly, an orifice (200 μm in diameter) punctured on a 25 μm thick Delrin wall that separates the cis (grounded) and the trans chambers of the flow cell was precoated with 1:10 hexadecane/pentane (Sigma-Aldrich). Then both chambers were filled with 1 mL of 3 M KCl solution buffered in 10 mM Tris-HCl (pH 8). To form a lipid bilayer membrane in the orifice, 20 μL (10 mg/mL) 1,2 diphytanoyl-sn-glycero-3-phosphocholine (Avanti Polar Lipids) dissolved in pentane (Sigma-Aldrich) was added to the cis side of chambers to allow self-assembly. Following this, electrical potential was applied to the trans side using Ag/AgCl electrodes and slowly ramped up to examine the stability of the membrane at ±200 mV. The membrane capacitance was maintained between 160-170 pF with various voltage bias values throughout each experiment.

To insert a single nanopore channel into the lipid bilayer, trans voltage was changed to 100 mV and a small amount (˜0.05 μg) of α-HL protein (Sigma-Aldrich) were added from a monomeric stock solution made in 3 M KCl to the cis compartment. To ensure consistency of testing conditions, the direction of each α-HL nanopore was examined by comparing the value of the channel current under positive and negative voltages after its insertion into the lipid bilayer. A properly inserted α-HL pore exhibits larger ionic current under a positive trans voltage than it is under a negative voltage, see FIG. 7 at (a). After a stable α-HL protein nanopore was inserted and confirmed by an open pore current, an analyte was added to the cis chamber at a bulk concentration of 200 μM from a 10 mM stock solution made in DMSO.

Data Collection and Analysis.

Ionic current recordings were collected using a patch clamp amplifier (Warner Instruments) with a built-in high-pass Bessel filter (cutoff: 5 kHz) at a holding potential of 100 mV. After sample addition to the cis chamber, magnetic stirring was used to disperse the sample before characteristic signal was recorded. The magnetic stirring was performed at the bottom of cis side to avoid any impact on the stability of the membrane and the nanopore, see FIG. 7 at (b) and (c). Each sample was measured in three replicates with a 30 min total duration. The ionic current was sampled at 100 kHz using a Digidata 1440A analog-to-digital converter (Molecular Devices) and processed with pClamp10 software (Molecular Devices).

A fresh α-HL protein nanopore was used for each replicate. The raw data was analyzed using an in-house Matlab based algorithm to find the current blockade and the dwell time of each eligible event, which are two commonly used properties for discriminating different molecules when they translocate nanopores. The current blockade that represents the capture of single molecules and their translocation through the nanopore is defined as I/I₀ (I=I₀−I_{b}, I_{b}: the average current measured with the molecules inside the pore; I₀: the average baseline current in absence of analytes). Dwell time (i.e. duration) represents the effective interaction time between nanopore and single molecule analyst, see FIG. 1 at (a). Results processed by the Matlab algorithm were confirmed by manual inspection.

Statistical Analysis.

To profile each analyte, current blockade was plotted against dwell time using Python. The python modules used for scatter plots and contour plots were Matplotlib and Seaborn's bivariate kernel density estimator. Contours were created according to the density of the data points in the logarithmic duration fractional blockade space, based on a kernel density function whereby every data point contributes a two-dimensional Gaussian to the cumulative contour, which was then normalized in z such that the entire volume of all of the contributing data integrated to one.

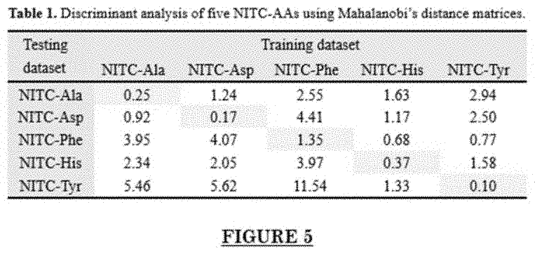

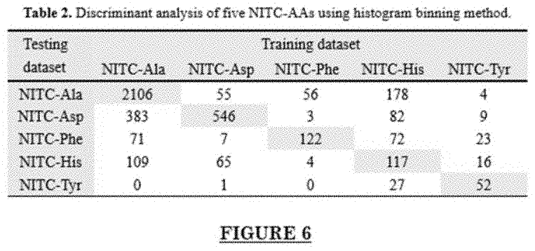

For discriminant analysis, multiple parallel experiments were performed for each NITC-AA respectively to collect >1000 events as the training datasets, which are used to calculate the similarity relationships with testing datasets of each NITC-AAs. All analyses were performed using Mahalanobis Distance matrices and histogram binning methods with an in-house MATLAB based program, see FIG. 5, Table 1, and FIG. 6, Table 2. The Mahalanobis distance is a measure of the distance between a point and a distribution. In this case, Mahalanobis distances for points in the testing datasets were calculated against each of the training datasets, and a smaller distance indicates a greater similarity. The same training datasets and testing datasets were used for the histogram binning technique. The training datasets were binned in two dimensions-residual current and dwell time measurements—with 50 bins in each dimension. Afterwards, the centroid (i.e. average residual current value and average dwell time of the entire cluster) was binned into the 2D histograms formed from the training data sets. An index was generated from the number of data points within the specific bin in the training dataset that would house the centroid of the testing data sets. A larger index represents greater similarity between the clusters. The binning was first conducted with the auto-binning capability in MATLAB, and then with 25, 50, 100, and 250 bins. After generating similar data tables for the different binning schemes and manually inspecting the binning schemes of the training set, it was found that the auto-binning capabilities did not generate consistent or sufficient bin numbers. For 25 bins, there was an inflated number of measurements in each bin and reduced the sensitivity of the technique. For the 250 bins, there was a deflated number of measurements in each bin and led to a greater number of anomalous measurements. Finally, we chose 50 bins to perform the comparison.

Molecular Modeling.

Geometry optimization of amino acids and their derivatives was calculated using Q-chem 4.3. Unrestricted B3LYP function was employed to describe our system, making use of the 6-31++G basis sets for C, H, O, N and S atoms. Solvent effects (KCl aqueous solution with a dielectric constant of 55) were included using the PCM implicit solvation model. The VDW radius was calculated by Multiwfn program, see Lu, T.; Chen, F. W. Multiwfn: A multifunctional wavefunction analyzer. Journal of Computational Chemistry 2012, 33, 580-592. 10.1002/jcc.22885, in which VDW surface is defined by the lengths of the three sides of the cube.

Amino Acids Derivatization and Characterization

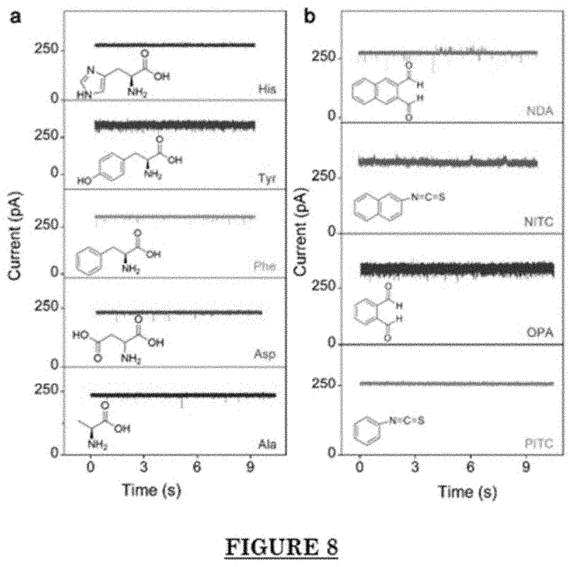

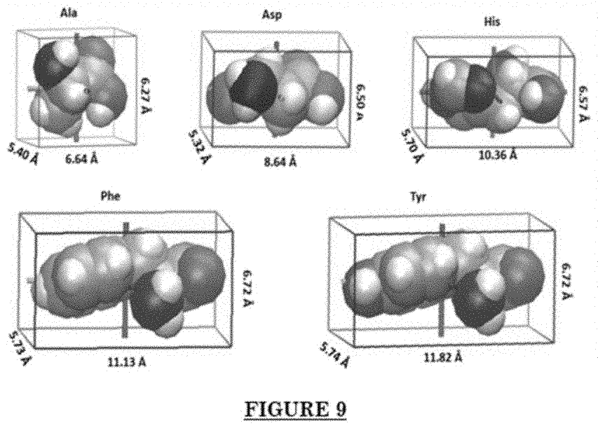

Five characteristic AAs with different size, charge, polarity, and hydrophobicity were selected in our initial study: Alanine (Ala), Phenylalanine (Phe), Tyrosine (Tyr), Aspartic Acid (Asp), and Histidine (His). Each unmodified AA was first analyzed using an α-HL nanopore for extended recording time, and no obvious current blockade signal was observed at applied potential bias, see FIG. 8 at (a). Similarly, no characteristic signal was recorded for the four derivatization reagents selected in this study (i.e. OPA, NDA, PITC, and NITC) through the nanopore, see FIG. 8 at (b). The low frequency of current blockade events for the selected amino acids and modifiers further demonstrates their weak interactions with the lumen of the α-HL nanopore, see FIG. 1 at (c), which can be attributed to the smaller Van der Waals radii of the amino acids (˜0.3-0.4 nm for all amino acid based on Spartan calculation, see FIG. 9, compared to the dimension of the constriction region of the α-HL nanopore (1.4 nm).

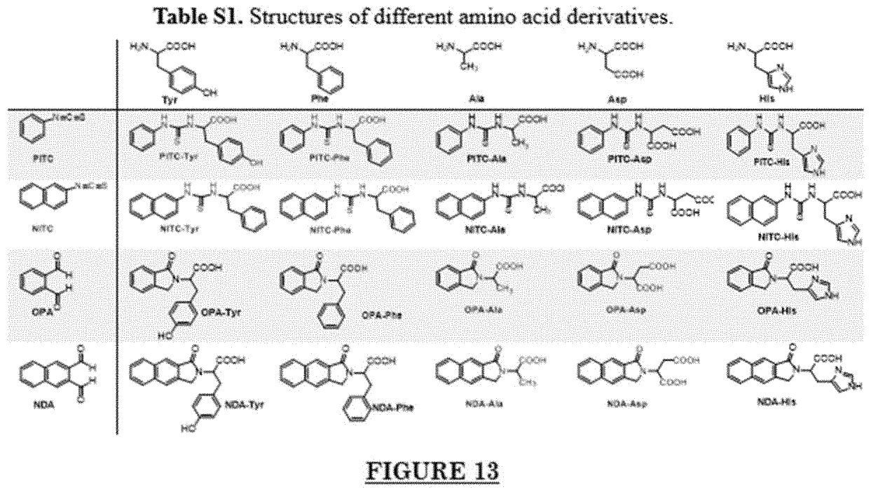

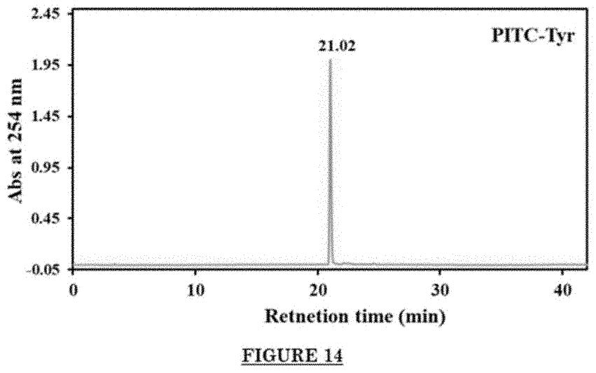

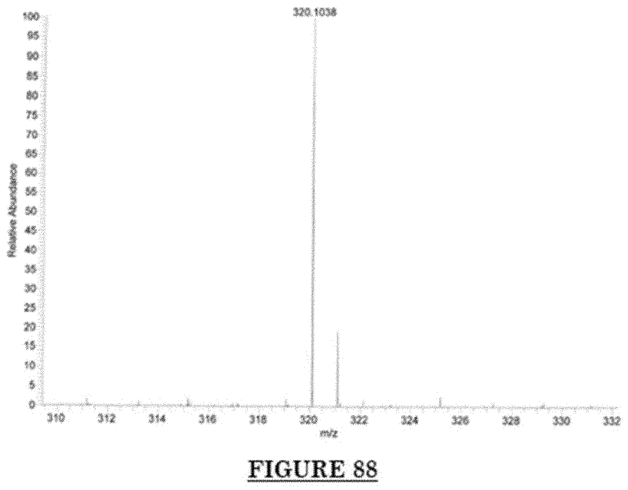

To increase effective interaction between the nanopore and analytes, the N-terminals of the aforementioned AAs were readily modified with PITC, OPA, NITC and NDA, respectively, see FIG. 1 at (b) and FIG. 13, Table 51. The structure and the purity (>99%) of all the derivatization products were confirmed by high-performance liquid chromatography (HPLC), mass spectrometry (MS), and nuclear magnetic resonance (NMR) analyses, see FIGS. 14-88. Purified amino acid derivtatives were employed in our nanopore analysis. As shown in FIG. 1 at (c), all four series of AA derivatives, especially the NITC and NDA derivatives, exhibited significant increase in the translocation event frequency through α-HL nanopores under the same conditions in comparison with the unmodified AAs, revealing improved interaction between AAs and the α-HL pore after derivatization.

Translocation Profiles of Amino Acid Derivatives

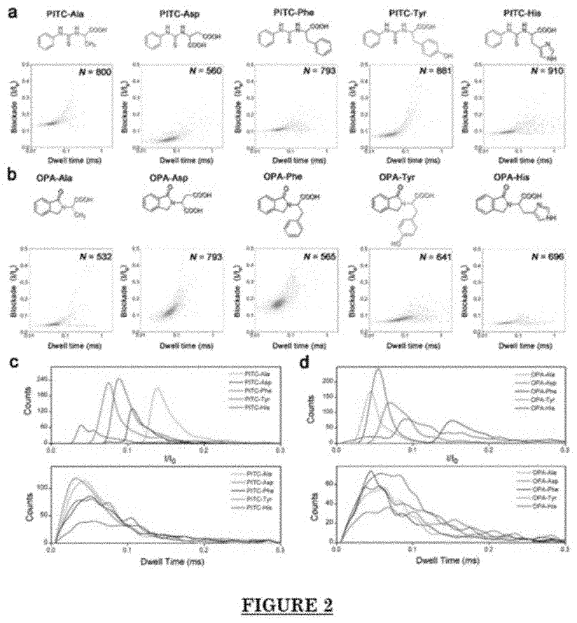

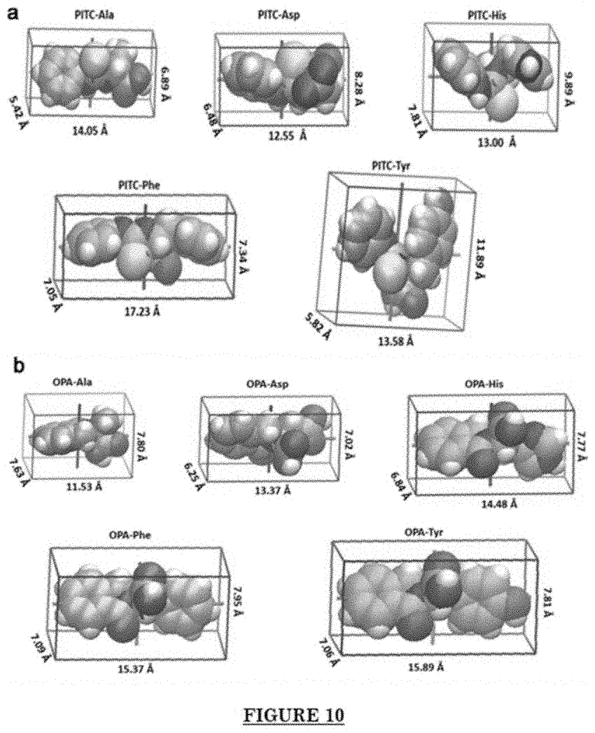

To assess the ability of PITC derivatization for distinguishing the five different AAs, a contour plot was generated for each of the five PITC-derivatives from its average current blockade and dwell time values to profile its translocation behavior, see FIG. 2 at (a). In combination with the frequency distribution of current blockade and dwell time, see FIG. 2 at (c), four out of the five different AAs were distinguishable from each other by their current blockade profiles, including Ala, Asp, Tyr, and His, even though they showed similar dwell time distribution. Unfortunately, the PITC-Phe derivative had significant overlaps with other AA derivatives in both current blockade and dwell time distributions, making it difficult to distinguish Phe from other AAs, especially Tyr and His. Similarly, OPA derivatization was tested. Although the formation of a five-membered ring between the AA and OPA should increase the rigidity of the AA derivatives, see FIG. 10, and may improve their interactions with the nanopore during translocation, the distinguishability across all AAs was reduced except for the OPA-Phe, see FIG. 2 at (b) and (d). FIG. 2 at (a) and (b) shows molecular structures and corresponding contour plots depicting the blockade (I/I₀) vs. dwell time distribution at the same applied potential for different AA derivatives: (a) PITC-derivatives, (b) OPA-derivatives. N value indicates the total number of stochastic signal events included in each analysis and (c) and (d) show translocation event frequency distribution of blockade (I/I₀) and dwell time for (c) PITC and (d) OPA derivatives.

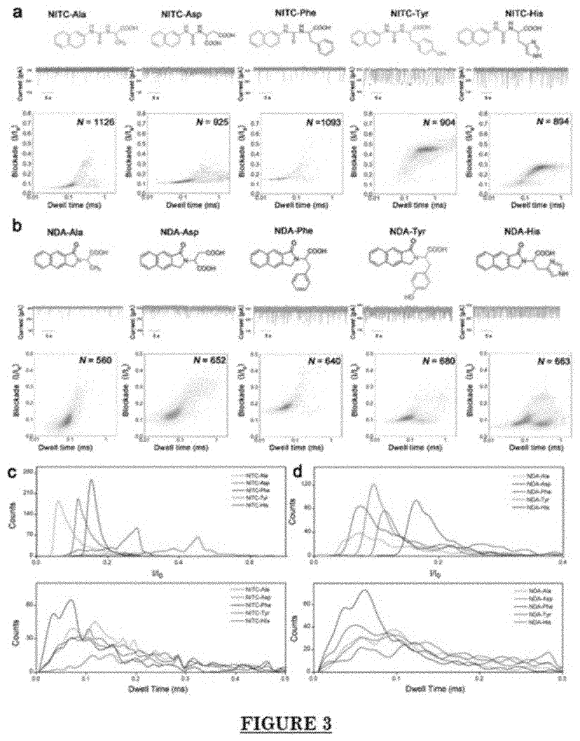

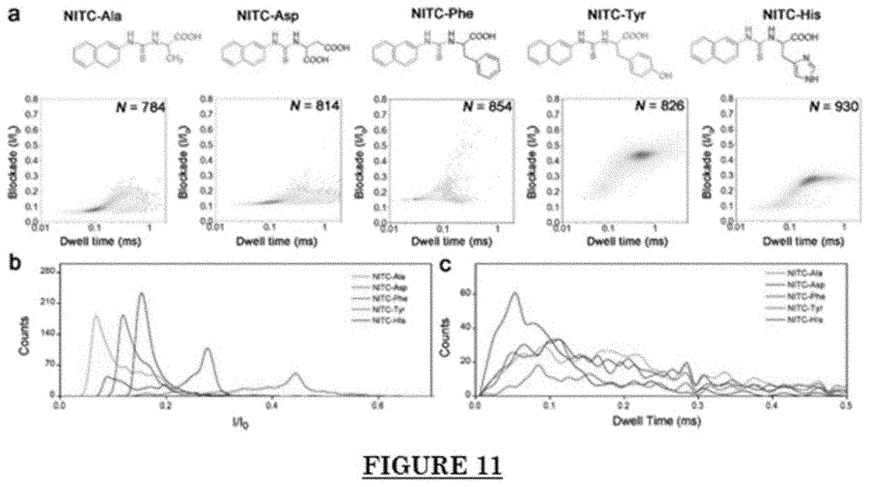

We next tested the NITC derivatization, which adds one more benzene ring to the structures. A clear differentiation among the five AAs could be observed through current blockade and dwell time analysis, see FIG. 3 at (a). In addition, stochastic event frequency was also increased significantly by NITC modification compared with PITC and OPA modifications, see FIG. 1 at (c). Despite the similar dwell time centered from 0.05-0.1 ms for NITC-Ala, NITC-Asp, and NITC-Phe, their current blockade (I/I₀) values concentrated at 0.06, 0.12, and 0.15 respectively with narrow distribution and minimal overlaps provided excellent distinguishability of these three AAs. NITC-Tyr and NITC-His derivatives exhibited larger current blockade values concentrated around 0.45 and 0.28 respectively with wider distribution. Together with their longer dwell times (>0.1 ms), these two NITC-derivatives can be effectively distinguished from NITC-Ala, NITC-Asp, and NITC-Phe, as well as from each other, see FIG. 3 at (c). In addition, the reproducibility of the contour profiles of NITC-derivatives, see FIG. 11, revealed a much more stable and uniform translocation behaviors, which is essential for future practical applications. With minimal data processing, NITC derivatization of each AAs could be readily distinguished. FIG. 3 shows at (a) and (b): from top to bottom: molecular structure, representative current trace of translocations, and corresponding contour plots of different (a) NITC-derivatives and (b) NDA-derivatives. N value indicates the total number of stochastic signal events included in each analysis and at (c) and (d): translocation event frequency distribution of blockade (I/I₀) and dwell time for (c) NITC-derivatives and (d) NDA-derivatives.

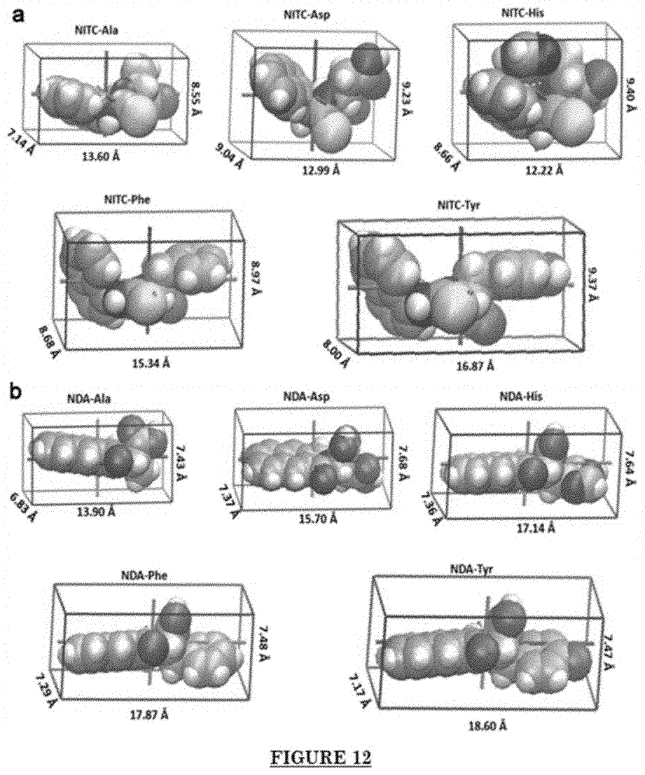

The current blockade and dwell time profiles of NDA-derivatives were similarly measured, see FIG. 3 at (b), which show wider distributions of events in general and significant higher signal overlaps than NITC-derivatives, see FIG. 3 at (d). This could be attributed to the much more rigid benzoisoindolone structure, see FIG. 12, resulting in smaller structural differences among different NDA-AAs. Interestingly, multiple peak areas were observed in the contour plots of NDA-Tyr and NDA-His, possibly due to multiple spatial orientations while passing through the nanopores. This phenomenon will be further investigated in our future studies.

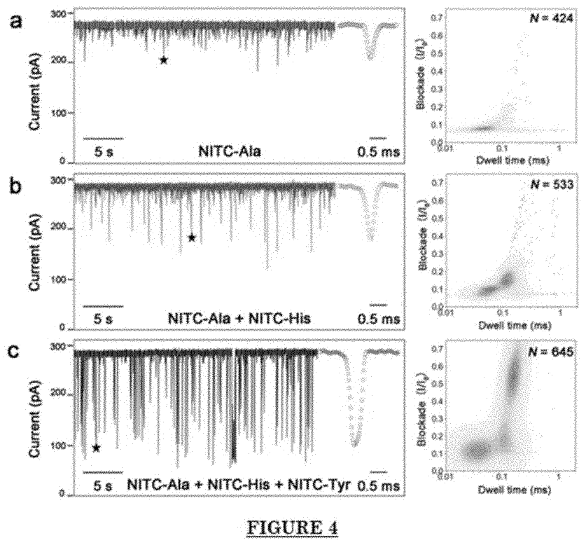

Finally, to assess the discriminatory power of our method for the differentiation of AAs, three different AAs (Ala, His, and Tyr) derivatized with NITC were added to the cis compartment of the nanop ore flow chamber sequentially at the same final concentration (200 μM) while translocation signals were recorded simultaneously. As shown in FIG. 4, the corresponding fingerprint of each compound can be clearly differentiated in the contour map of the mixtures. Despite the interaction and competitive translocation between different derivatives, each AA derivative exhibited reproducible translocation behavior (current blockade vs. dwell time distributions) comparing to its fingerprint obtained separately in our previous experiments, see FIG. 3 at (a). However, fingerprints of NITC-Ala, NITC-Asp, and NITC-Phe would easily merge into each other on the qualitative contour plot. To efficiently identify AAs, quantitative biostatistics analysis with excellent inter-pore reproducibility is needed to recogonize AA derivatives from unknown samples using their established translocation profiles. Discriminant analyses of five NITC-AAs were performed using both Mahalanobis distance matrices and histogram binning method. A combination of multiple replicates of signal event clusters were chosen as the training datasets based on their experimental reliability and reproducibility. The Mahalanobi's distances were calculated for the testing datasets against each of the training datasets. A smaller distance indicates a greater similarity, see FIG. 5, Table 1. The same training datasets and testing datasets were used for the histogram binning technique, and 50 bins were used for the calculation. A larger number indicates a greater similarity, see FIG. 6, Table 2. For each sample, at least 500 events were analyzed. FIG. 4 shows discrimination of each AA in a mixture sample. After a single α-HL nanopore was obtained in the working buffer (3M KCl, 10 mM Tris-HCl, pH 8.0), 200 μM NITC-Ala, 200 μM NITC-His, and 200 μM NITC-Tyr were sequentially added to the cis compartment with 5 min intervals FIG. 4 at (a)-(c) shows representative current trace of translocations (Left) obtained and contour plots depicting current blockade vs. dwell time distributions of all translocation events (Right) for (a) NITC-Ala, (b) NITC-Ala and NITC-His, and (c) NITC-Ala, NITC-His, NITC-Tyr. The stars denote the typical spike signals shown in the inset.

### DISCUSSION

Revealing the primary sequence of a protein or peptide is essential to its identification and function. Traditionally, the most common method for protein sequencing is MS, a technique that involves fractionating the protein into many smaller peptides and then obtaining the mass-to-charge ratio of each new peptide from the mass spectrometer. However, sequencing is sometimes impossible with this technology due to low abundance of precursor peptide and poor fractionation efficiency. The sensitivity of MS also varies among different analytes and between instrument models. Matrix-assisted laser desorption/ionization-time of flight (MALDI-TOF) MS suffers from significant reduced sensitivity on samples with high concentration of salts. Although many high-resolution MS have been developed recently and the combination with high performance liquid chromatography may improve the sensitivity, it is still a laborious process to profile the complete sequence of an unknown protein. In addition, MS instruments are too costly and complex to be developed into portable devices. A portable, accurate, and easy-to-use protein sequencer will engender future implications in personalized medicine, especially in self-testing, resource-limited settings, disease outbreaks, and novel theranostics concepts. Resistive pulse sensing using biological nanopores shows atomic precision due to the extremely small dimension of their sensing regions in the pore lumen (1˜4 nm), and has been demonstrated with excellent sensitivity and accuracy in DNA and RNA sequencing. Recently, the focus of efforts has been directed towards amino acid identification and sequencing of proteins and peptides, which holds great promise for the advancement of proteomics. Comparing to MS, the nanopore technology has several advantages: (1) long-reads that are not limited by precursor peptide fractionation; (2) high tolerance to contaminations such as salts and polymers; (3) simplicity and cost efficiency.

Pioneering studies have demonstrated that a nanopore is able to detect certain single AA, and differentiate certain peptides with one AA difference in length. However, sequencing of random peptides with single AA resolution is still extremely challenging to realize, likely due to the zwitterionic form, the non-uniform translocation rate, as well as low signal-to-noise ratio and low distinguishability of most AAs caused by the mismatch between the diameter of AAs (0.6-0.8 nm) and the nanopore (1.4-3 nm). Inspired by the Edman degradation, the current disclosure developed a new nanopore method to identify a single AA using its N-terminal derivative as a surrogate. Four derivative regents were employed in this study to modify five AAs. Our results indicate that the derivatization afforded a fingerprint on stochastic signals when each AA translocating the α-HL nanopore due to the increased interaction. Importantly, these derivatization methods were efficient and reproducible under simple one-pot mild reaction conditions, affording a reliable strategy for the formation of a structurally diverse array of AA derivatives.

The current disclosure assessed four series of AA derivatives for their translocation behaviors through the α-HL nanopore, including PITC-derivatives, OPA-derivatives, NITC-derivatives, and NDA-derivatives of five different AAs (Ala, Asp, Phe, Tyr, and His) with various polarity, charge and size, representing different types of twenty AAs. Significantly increased translocation event frequency of derivatives comparing to unmodified AAs indicates enhanced interaction between the nanopore lumen and analytes. Detailed investigation on the translocation signals of all derivatives by estimating the distribution of current blockade and dwell time revealed best distinguishability among Ala, Asp, Phe, Tyr, and His by the NITC-derivatization. As confirmed by the molecular structure modeling for each AA and AA derivative, see FIGS. 9, 10, and 12, some derivatives with larger cubic volume, such as NITC-His and NITC-Tyr, produced deep current blockades even larger than that of ssDNAs. However, exceptions such as NITC-Phe indicates that other interactions (i.e. charge, polarity, intra- and inter-molecular interactions etc.) also play a role during the translocation. Although it is difficult to quantitatively determine the impact of these interactions on the event frequency and the changing trend of fingerprint of each derivative at the moment, further investigations using suitable mutant hemolysin nanopores and other types of biological nanopores may provide more insights.

The current disclosure clearly demonstrates that derivatization is a feasible way to identify single AAs with biological nanopores, we do recognize the overall complexity of protein sequencing using nanopore. Although the technology has been proven successful for nucleic acid sequencing, it is considerably more challenging to differentiate 20 AAs than 4 nucleotides. In addition to sensitivity enhancement via different modifications to nanopores and analytes through biochemistry methods, more advanced data analysis technology (e.g. machine learning, pattern recognition, etc.) is in urgent need to improve resolution through novel characteristics other than the traditional blockade and dwell time from the stochastic signals. Similar to the MS technology, the data readout from nanopore also needs sophisticated bioinformatics database and algorithm to be interpreted into sequences and protein identifications. Therefore, the success of nanopore based protein sequencing no doubt requires multidisciplinary efforts.

Inspired by the protein ladder sequencing technique and the identification of single AA differences in length of peptides, the current disclosure provides a potential “Sequencing-by-Hydrolysis” method, in which a nanopore will be used to identify the N-terminal AA of each peptide fragment in a peptide ladder generated from a peptide analyte, and then bioinformatics methods will be applied to reconstitute its full-length sequence.

The current disclosure has demonstrated that N-terminus derivatization is an effective way to differentiate individual AAs using α-HL nanopore technology. Among four derivatization reagents applied in our work, NITC-derivatization of five typical AAs afforded significantly enhanced distinguishability based on the translocation signals. While we are working on developing more effective N-terminus modification strategies and optimizing the modifier's structure, more advanced data analysis technology is in urgent need to improve resolution through novel characteristics other than the traditional blockade and dwell time from the stochastic signals. Finally, further simulation work is undergoing to better model the conformational changes of each derivatives inside the lumen of the α-HL, and to understand the complexity of the interactions between each amino acid derivative and the lumen of the α-HL protein.

Supporting Information

1) Change of open pore current value when changing voltage direction after one α-HL nanopore inserted into the lipid membrane; and the current trace during the stirring process. 2) Representative current trace of the translocation of the raw materials through α-HL nanopores; 3) The space-filling structures of different AAs and AA derivatives calculated using the Q-chem 4.3 software package; 4) Reproducibility study of NITC-derivatives; 5) Table including structure of different amino acid derivatives. 6) General procedure and characterization of different derivatives.

FIG. 7 shows at (a) change of open pore current value when changing voltage (100 mV) direction after one α-HL nanopore is inserted into the lipid membrane. The inset is a schematic illustration of the direction of an inserted α-HL protein corresponding to the recorded current trace, at (b) current trace of the lipid membrane with capacitance of 160 pF during the stirring process, and at (c) current trace of translocations observed by one α-HL nanopore on the lipid membrane after adding the sample and stirring for a few seconds.

FIG. 8 shows representative current trace of translocations of the raw materials through α-HL nanopores (final concentration 200 μM) at (a) amino acids, and at (b) derivatization reagents. The structures of each chemicals and the code names are presented as insets. Data were acquired in a buffer of 3 M KCl and 10 mM Tris (pH 8.0) with the transmembrane potential held at +100 mV.

FIG. 9 shows space-filling structures of five selected amino acids based on molecular modeling with Q-chem 4.3 software package.

FIG. 10 shows space-filling structures of (a) PITC derivatized AAs and (b) OPA derivatized AAs calculated using the Q-chem 4.3 software package.

FIG. 11 shows at (a) from top to bottom: molecular structure and corresponding contour plots of different NITC-derivatives in repeated experiments, and at (b) and (c): translocation event frequency distributions of current blockade (b) and dwell time (c) of each NITC-derivative, respectively.

FIG. 12 shows Space-filling structures of (a) NITC derivatized AAs and (b) NDA derivatized AAs calculated using the Q-chem 4.3 software package.

FIG. 13 shows Table 51, chemical structures of different amino acid derivatives.

Detailed Procedure for Preparing Different Derivatives

Derivatization of amino acid with phenyl isothiocyanate (PITC): PITC (4 mmol), amino acid (4.8 mmol), Na₂CO₃ (0.1 M, 10 mL) and acetonitrile (20 mL) were stirred under reflux condition overnight. The resulting precipitate was collected, washed with 1 M HCl and methanol, and dried to afford the corresponding product. The average yields are 70-85%.

Derivatization of amino acid with 2-naphthaleneisothiocyanate (NITC): NITC (1 mmol), amino acid (1.1 mmol), Na₂CO₃ (0.1 M, 4 mL) and acetonitrile (8 mL) under reflux condition overnight. The resulting precipitate was collected, washed with 1 M HCl and methanol, and dried to afford the corresponding product. The average yields are 60-81%.

Derivatization of amino acid with o-phthalaldehyde (OPA): OPA (3.6 mmol), amino acid (3 mmol), trifluoroacetate (4.2 mmol) and acetonitrile (30 mL) were stirred under reflux condition for 3 h. The precipitate was washed with acetonitrile to afford the corresponding product. The average yields are 50-70%.

Derivatization of amino acid with 2,3-naphthalenedicarboxaldehyde (NDA): NDA (0.7 mmol), amino acid (0.6 mmol), trifluoroacetate (0.8 mmol) and acetonitrile (10 mL) were stirred under reflux condition for 3 h. The precipitate was washed with acetonitrile to afford the corresponding product. The average yields are 45-79%.

Characterization of Derivatives by High Performance Liquid Chromatography (HPLC)

Samples were desolved in DMSO/methanol (5:95), injected 5 uL in an Agilent 1100 HPLC equipped with a ZORBAX SB-C18 column. HPLC parameters were as follows: 25° C.; solvent A, 1% acetic acid in water; solvent B, methanol; gradient, 10% B for 2 min; then, from 10% B to 100% B over 32 min; then, from 100% B to 10% B over 10 min, flow rate, 0.5 mL/min. Detection of the products was by UV absorbance at 254 nm or 286 nm.

FIG. 14 shows HPLC spectrum of PITC-Tyr.

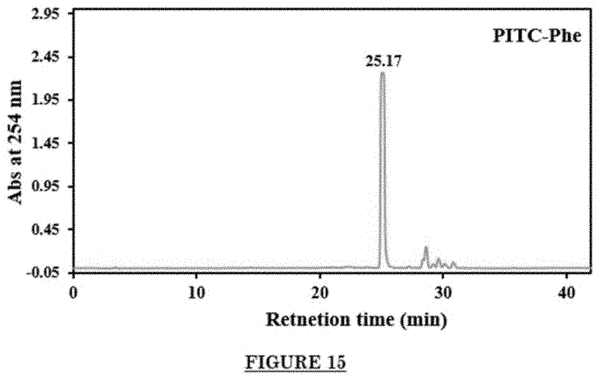

FIG. 15 shows HPLC spectrum of PITC-Phe.

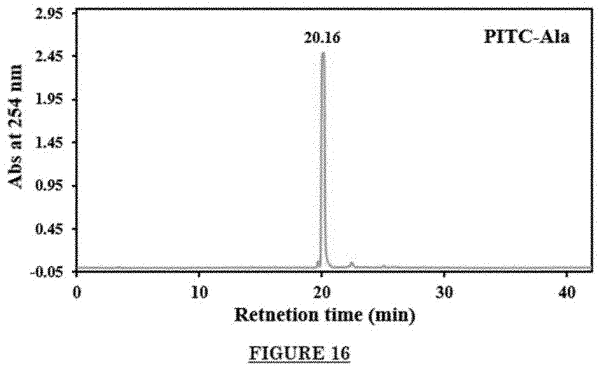

FIG. 16 shows HPLC spectrum of PITC-Ala.

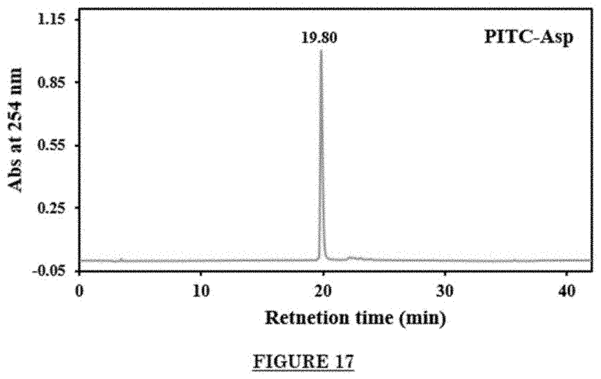

FIG. 17 shows HPLC spectrum of PITC-Asp.

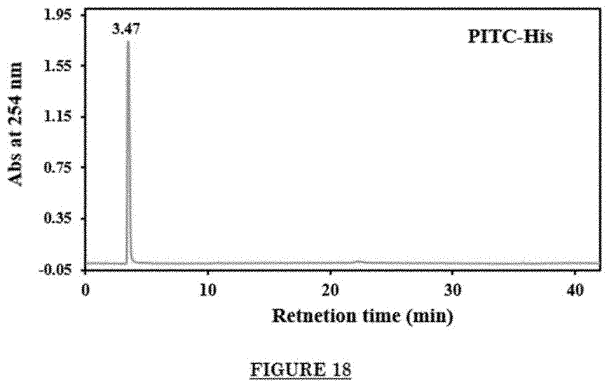

FIG. 18 shows HPLC spectrum of PITC-His.

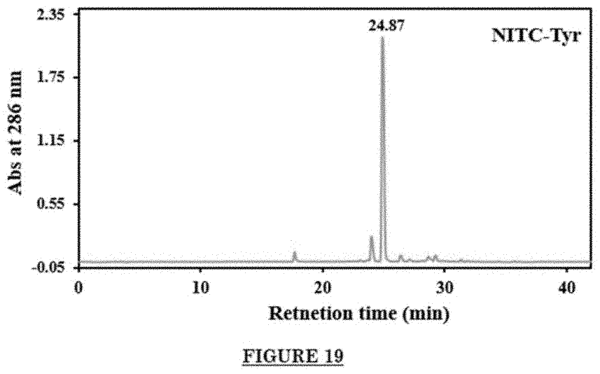

FIG. 19 shows HPLC spectrum of NITC-Tyr.

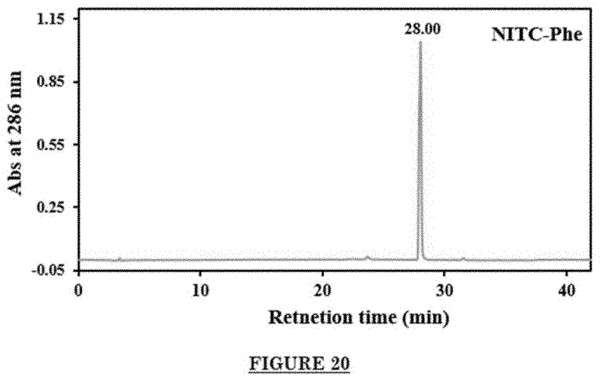

FIG. 20 shows HPLC spectrum of NITC-Phe.

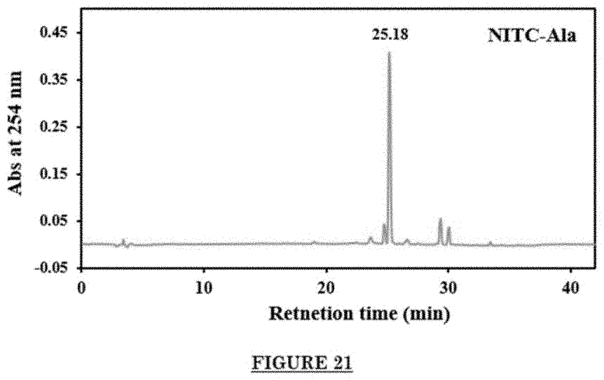

FIG. 21 shows HPLC spectrum of NITC-Ala.

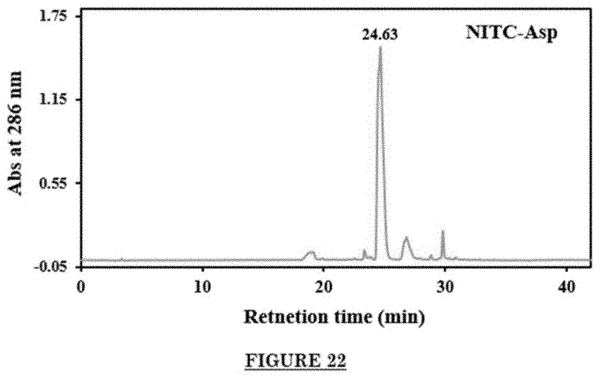

FIG. 22 shows HPLC spectrum of NITC-Asp.

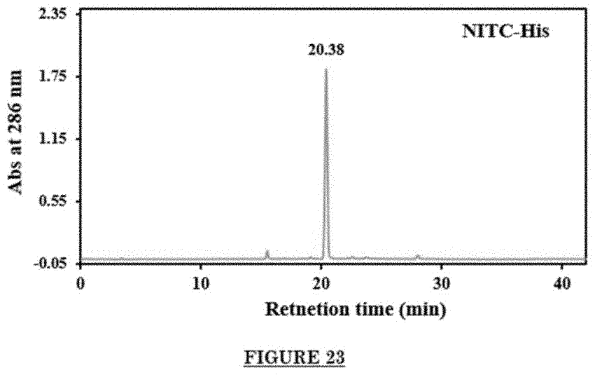

FIG. 23 shows HPLC spectrum of NITC-His.

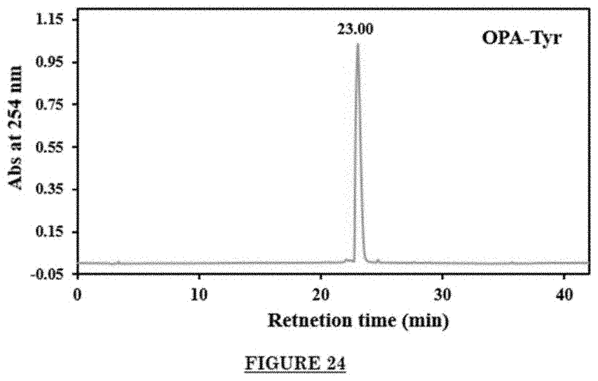

FIG. 24 shows HPLC spectrum of OPA-Tyr.

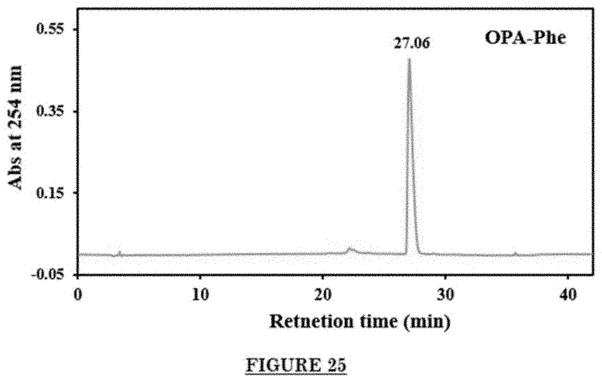

FIG. 25 shows HPLC spectrum of OPA-Phe.

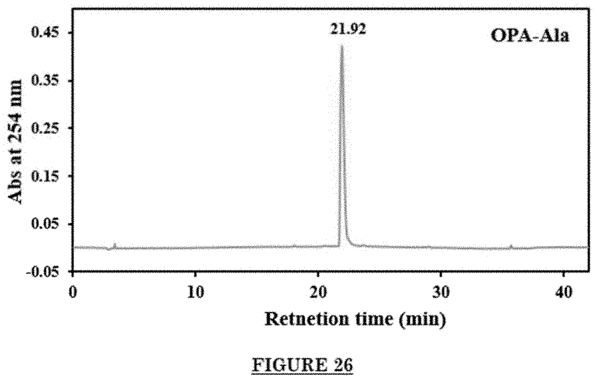

FIG. 26 shows HPLC spectrum of OPA-Ala.

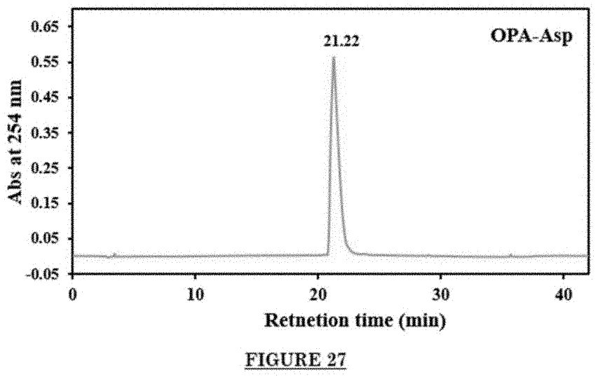

FIG. 27 shows HPLC spectrum of OPA-Asp.

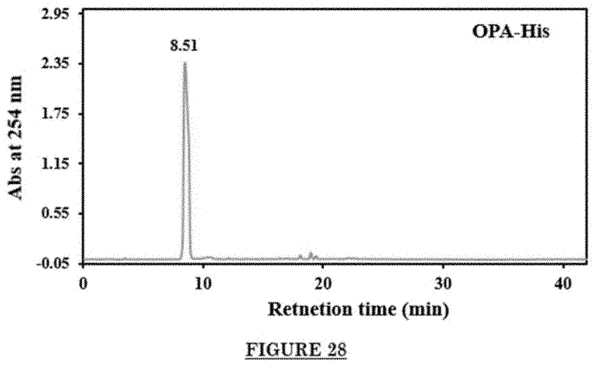

FIG. 28 shows HPLC spectrum of OPA-His.

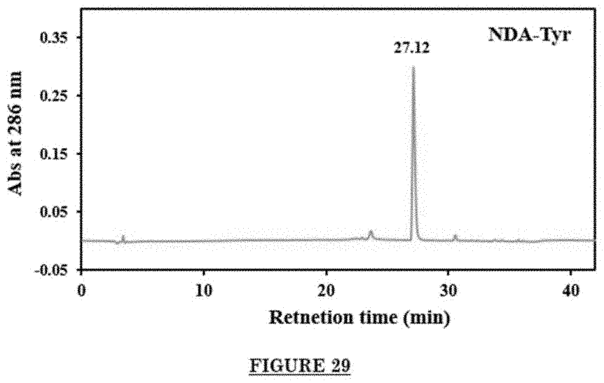

FIG. 29 shows HPLC spectrum of NDA-Tyr.

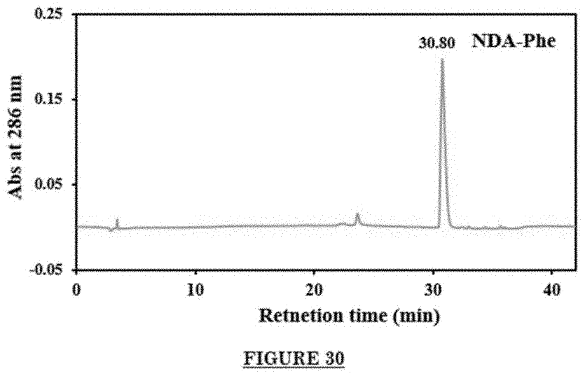

FIG. 30 shows HPLC spectrum of NDA-Phe.

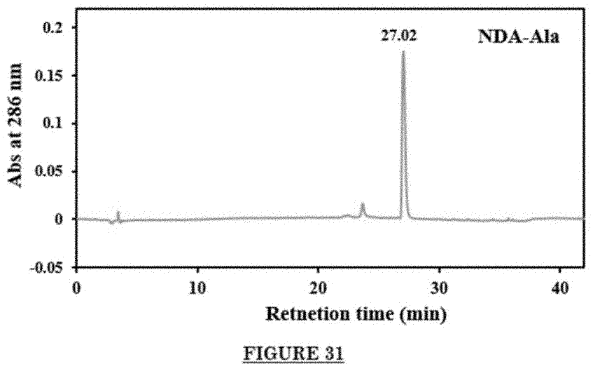

FIG. 31 shows HPLC spectrum of NDA-Ala.

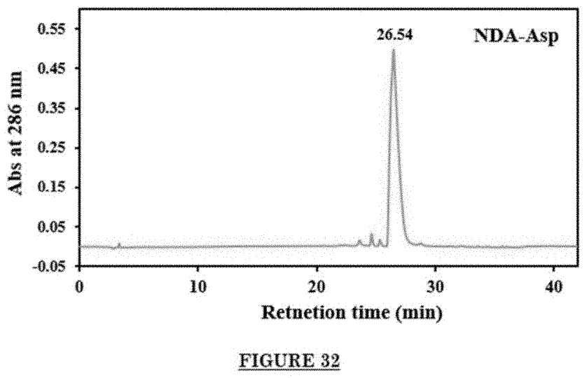

FIG. 32 shows HPLC spectrum of NDA-Asp.

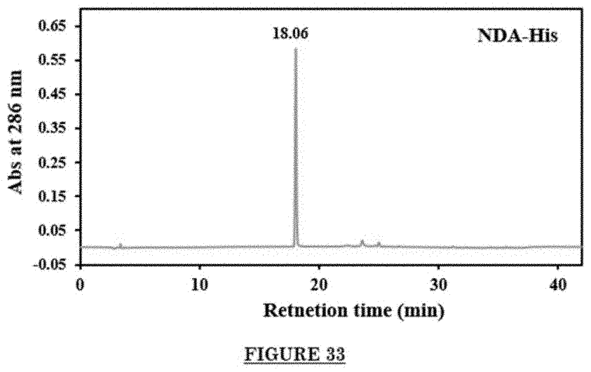

FIG. 33 shows HPLC spectrum of NDA-His.

Characterization of Derivatives by High Resolution Mass Spectrometry (HRMS) and ¹H&¹³C Nuclear Magnetic Resonance (NMR)

### (Phenylcarbamothioyl)tyrosine

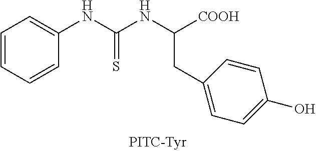 [SMILES: O=C(O)C(Cc1ccc(O)cc1)NC(=S)Nc1ccccc1]

¹H NMR (DMSO-d₆, 400 MHz): δ 10.68 (s, 1H), 9.33 (s, 1H), 7.42-7.37 (m, 3H), 6.99 (d, 2H, J=8.4 Hz), 6.82 (dd, 2H, J₁=7.7 Hz, J₂=1.8 Hz), 6.69 (d, 2H, J=8.5 Hz), 4.69-4.67 (m, 1H), 3.05-2.69 (m, 2H). ¹³C NMR (DMSO-d₆, 100 MHz): δ 182.2, 173.6, 156.4, 133.2, 130.8, 128.6, 128.5, 128.4, 124.3, 114.9, 60.2, 35.3. HRMS: [M+H]⁺ m/z calcd for C₁₆H₁₆N₂O₃SH⁺, 317.0954; found, 317.0953.

### (Phenylcarbamothioyl)phenylalanine

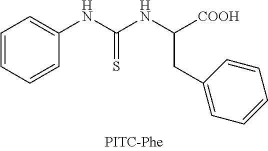 [SMILES: O=C(O)C(Cc1ccccc1)NC(=S)Nc1ccccc1]

¹H NMR (DMSO-d₆, 400 MHz): δ10.61 (s, 1H), 7.41-7.36 (m, 3H), 7.35-7.29 (m, 3H), 7.22-7.20 (m, 2H), 6.79-6.76 (m, 2H), 4.79-4.77 (m, 1H), 3.13 (d, 2H, J=4.5 Hz). ¹³C NMR (DMSO-d₆, 100 MHz): δ 182.2, 173.5, 134.5, 133.2, 129.8, 128.7, 128.6, 128.4, 124.2, 127.1, 60.2, 36.1. HRMS: [M+H]⁺ m/z calcd for C₁₆H₁₆N₂O₂SH⁺, 301.1005; found, 301.1005.

### (Phenylcarbamothioyl)alanine

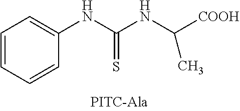

¹H NMR (DMSO-d₆, 400 MHz): δ10.54 (s, 1H), 7.51-7.40 (m, 3H), 7.31-7.28 (m, 2H), 4.47 (q, 1H, J=7.1 Hz), 1.40 (d, 2H, J=7.1 Hz). ¹³C NMR (DMSO-d₆, 100 MHz): δ 182.0, 175.0, 133.5, 128.8, 128.7, 128.5, 55.1, 16.2. HRMS: [M+H]⁺ m/z calcd for C₁₀H₁₂N₂O₂SH⁺, 225.0692; found, 225.0692.

### (Phenylcarbamothioyl)aspartic acid

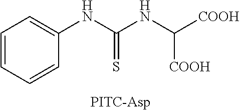 [SMILES: O=C(O)C(NC(=S)Nc1ccccc1)C(=O)O]

¹H NMR (DMSO-d₆, 400 MHz): δ10.41 (s, 1H), 7.50-7.46 (m, 2H), 7.44-7.40 (m, 1H), 7.26-7.24 (m, 2H), 4.56 (t, 1H, J=4.4 Hz), 2.93/2.78 (q, 2H, J=7.3 Hz). ¹³C NMR (DMSO-d₆, 100 MHz): δ 183.1, 174.1, 170.7, 133.8, 128.7 (2C), 128.5, 55.8, 34.8. HRMS: [M−H]⁻ m/z calcd for C₁₁H₁₁N₂O₄S⁻, 267.0445; found, 267.0452.

### (Phenylcarbamothioyl)histidine

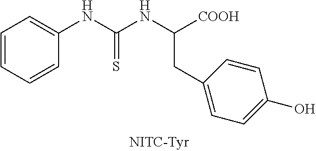 [SMILES: O=C(O)C(Cc1ccc(O)cc1)NC(=S)Nc1ccccc1]

¹H NMR (DMSO-d₆, 400 MHz): δ 11.88 (s, 1H), 10.41 (s, 1H), 7.57 (s, 1H), 7.48-7.39 (m, 3H), 7.15 (d, 2H, J=7.4 Hz), 6.86 (s, 1H), 4.65 (t, 1H, J=4.7 Hz), 3.07 (d, 2H, J=4.7 Hz). ¹³C NMR (DMSO-d₆, 100 MHz): δ 183.0, 174.4, 135.3, 134.1, 133.1, 129.2, 129.1, 128.9, 116.2, 59.6, 29.3. HRMS: [M−H]⁻ m/z calcd for C₁₃H₁₃N₄O₂S⁻, 289.0765; found, 289.0770.

### (Naphthalen-2-ylcarbamothioyl)tyrosine

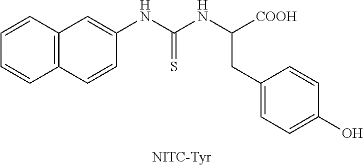 [SMILES: O=C(O)C(Cc1ccc(O)cc1)NC(=S)Nc1ccc2ccccc2c1]

¹H NMR (DMSO-d₆, 400 MHz): δ 10.06 (s, 1H), 9.62 (s, 1H), 7.94-7.88 (m, 2H), 7.84 (d, 1H, J=7.0 Hz), 7.57-7.55 (m, 2H), 7.25 (s, 1H), 7.02 (d, 2H, J=7.3 Hz), 6.86 (d, 1H, J=8.6 Hz), 6.73 (d, 2H, J=7.1 Hz), 4.70 (s, 1H), 3.05 (d, J=2.5 Hz, 2H). ¹³C NMR (DMSO-d₆, 100 MHz): δ 182.8, 174.4, 156.9, 132.89, 132.87, 131.4, 131.1, 128.8, 128.2, 128.1, 127.8, 127.6, 127.2, 126.5, 124.8, 115.4, 61.1, 35.8. HRMS: [M−H]⁻ m/z calcd for C₂₀H₁₈N₂O₃S⁻, 365.0965; found, 365.0965.

### (Naphthalen-2-ylcarbamothioyl)phenylalanine

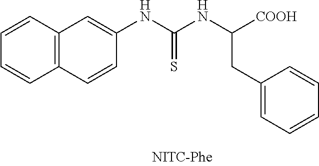 [SMILES: O=C(O)C(Cc1ccccc1)NC(=S)Nc1ccc2ccccc2c1]

¹H NMR (DMSO-d₆, 400 MHz): δ10.68 (s, 1H), 7.96-7.86 (m, 3H), 7.61-7.53 (m, 2H), 7.38-7.32 (m, 4H), 7.26-7.23 (m, 2H), 6.85 (dd, 1H, J₁=10.6 Hz, J₂=6.8 Hz), 4.83 (t, 1H, J=4.3 Hz), 3.16 (d, 2H, J=4.5 Hz). ¹³C NMR (DMSO-d₆, 100 MHz): δ 182.8, 174.1, 135.0, 133.0, 132.9, 131.1, 130.0, 128.69, 128.67, 128.3, 128.1, 127.8, 127.6, 127.4, 127.1, 126.5, 60.8, 36.7. HRMS: [M−H]⁻ m/z calcd for C₂₀H₁₇N₂O₂S⁻, 349.1016; found, 349.1011.

### (Naphthalen-2-ylcarbamothioyl)alanine

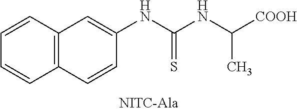 [SMILES: CC(NC(=S)Nc1ccc2ccccc2c1)C(=O)O]

¹H NMR (DMSO-d₆, 400 MHz): δ 10.62 (s, 1H), 8.03-8.00 (m, 3H), 7.89 (d, 1H, J=1.7 Hz), 7.64-7.57 (m, 2H), 7.43 (q, 1H, J=3.6 Hz), 4.57-4.50 (m, 1H), 1.45 (d, 3H, J=7.1 Hz). ¹³C NMR (DMSO-d₆, 100 MHz): δ182.6, 175.6, 133.1, 133.0, 131.4, 128.7, 128.4, 128.2, 128.1, 127.4, 127.1, 127.0, 55.7, 16.7. HRMS: [M+H]⁺ m/z calcd for C₁₄H₁₄N₂O₂SH⁺, 275.0849; found, 275.0849.

### (Naphthalen-2-ylcarbamothioyl)aspartic acid

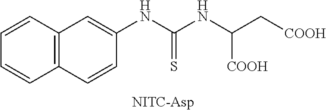 [SMILES: O=C(O)CC(NC(=S)Nc1ccc2ccccc2c1)C(=O)O]

¹H NMR (DMSO-d₆, 400 MHz): δ 12.8 (s, 1H), 10.41 (s, 1H), 8.03-7.97 (m, 3H), 7.83 (d, 1H, J=1.8 Hz), 7.64-7.57 (m, 2H), 7.40 (q, 1H, J=3.5 Hz), 4.68 (q, 1H, J=4.4 Hz), 3.00/2.84 (q, 2H, J=7.3 Hz). ¹³C NMR (DMSO-d₆, 100 MHz): δ 183.2, 174.2, 170.8, 132.7, 132.5, 131.3, 128.3, 128.0, 127.7, 127.6, 126.9, 126.6, 126.4, 55.9, 34.8. HRMS: [M+H]⁺ m/z calcd for C₁₅H₁₄N₂O₄SH⁺, 319.0747; found, 319.0747.

### (Naphthalen-2-ylcarbamothioyl)histidine

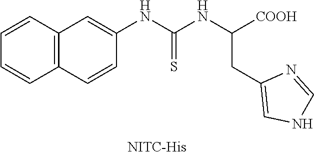 [SMILES: O=C(O)C(Cc1c[nH]cn1)NC(=S)Nc1ccc2ccccc2c1]

¹H NMR (DMSO-d₆, 400 MHz): δ 10.23 (s, 1H), 8.29 (d, 1H, J=8.7 Hz), 8.24 (s, 1H), 8.00 (s, 1H), 7.84 (d, 2H, J=8.7 Hz), 7.80 (d, 1H, J=8.0 Hz), 7.51-7.40 (m, 3H), 5.18 (q, 1H, J=5.0 Hz), 3.19-3.09 (m, 2H). ¹³C NMR (DMSO-d₆, 100 MHz): δ 180.5, 172.4, 136.7, 134.3, 133.2, 131.6, 130.0, 128.2, 127.5, 127.4, 126.4, 125.2, 123.2, 119.2, 116.5, 56.4, 27.8. HRMS: [M−H]⁻ m/z calcd for C₁₇H₁₅N₄O₂S⁻, 339.0921; found, 339.0917.

### 3-(4-Hydroxyphenyl)-2-(1-oxoisoindolin-2-yl)propanoic acid

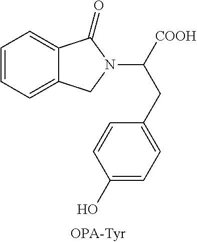 [SMILES: O=C(O)C(Cc1ccc(O)cc1)N1Cc2ccccc2C1=O]

¹H NMR (DMSO-d₆, 400 MHz): δ 13.08 (s, 1H), 9.20 (s, 1H), 7.64-7.55 (m, 3H), 7.49-7.43 (m, 1H), 7.04 (d, 2H, J=8.4 Hz), 6.60 (d, 2H, J=8.4 Hz), 5.05 (q, 1H, J=5.4 Hz), 4.44 (s, 2H), 3.31-3.04 (m, 2H). ¹³C NMR (DMSO-d₆, 100 MHz): δ 172.6, 168.3, 156.3, 142.4, 132.1, 132.0, 129.9, 128.4, 127.8, 123.9, 123.3, 115.6, 55.5, 47.8, 34.4. HRMS: [M+H]⁺ m/z calcd for C₁₇H₁₅NO₄H⁺, 298.1074; found, 298.1074.

### 2-(1-Oxoisoindolin-2-yl)-3-phenylpropanoic acid

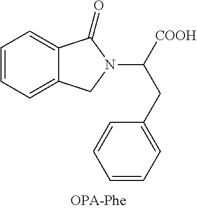 [SMILES: O=C(O)C(Cc1ccccc1)N1Cc2ccccc2C1=O]

¹H NMR (DMSO-d₆, 400 MHz): δ 7.64-7.56 (m, 3H), 7.48-7.44 (m, 1H), 7.28-7.21 (m, 4H), 7.16-7.13 (m, 1H), 5.16 (q, 1H, J=5.4 Hz), 4.49-4.40 (M, 2H), 3.43-3.18 (m, 2H). ¹³C NMR (DMSO-d₆, 100 MHz): δ 172.6, 168.3, 156.3, 142.4, 132.1, 132.0, 129.9, 128.4, 127.8, 123.9, 123.3, 115.6, 55.5, 47.7, 34.4. HRMS: [M+H]⁺ m/z calcd for C₁₇H₁₅NO₃H⁺, 282.1125; found, 282.1123.

### 2-(1-Oxoisoindolin-2-yl)propanoic acid

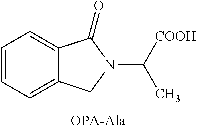

¹H NMR (DMSO-d₆, 400 MHz): δ 12.9 (s, 1H), 7.71 (d, J=7.5 Hz, 1H), 7.66-7.61 (m, 2H), 7.54-7.48 (m, 1H), 4.84 (q, 1H, J=7.5 Hz), 4.52 (dd, 2H, J₁=17.3 Hz, J₂=7.5 Hz), 1.51 (d, 3H, J=7.5 Hz). ¹³C NMR (DMSO-d₆, 100 MHz): δ173.5, 168.0, 142.6, 132.4, 132.0, 128.4, 124.0, 123.3, 49.6, 47.3, 15.8. HRMS: [M+H]⁺ m/z calcd for C₁₁H₁₁NO₃H⁺, 206.0812; found, 206.0812.

### 2-(1-Oxoisoindolin-2-yl)succinic acid

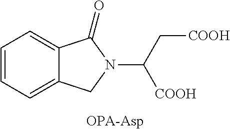 [SMILES: O=C(O)CC(C(=O)O)N1Cc2ccccc2C1=O]

¹H NMR (DMSO-d₆, 400 MHz): δ 12.7 (s, 1H), 7.71 (d, 1H, J=7.5 Hz), 7.66-7.61 (m, 2H), 7.54-7.48 (m, 1H), 5.10 (dd, 1H, J₁=6.4 Hz, J₂=1.5 Hz), 4.49 (s, 2H), 3.06-2.86 (m, 2H). ¹³C NMR (DMSO-d₆, 100 MHz): δ 172.2, 171.8, 168.0, 142.5, 132.2, 132.1, 128.4, 124.0, 123.4, 51.4, 48.4, 35.0. HRMS: [M+H]⁺ m/z calcd for C₁₂H₁₁NO₅H⁺, 250.0710; found, 250.0711.

### 3-(1H-imidazol-4-yl)-2-(1-oxoisoindolin-2-yl)propanoic acid

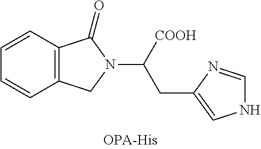 [SMILES: O=C(O)C(Cc1c[nH]cn1)N1Cc2ccccc2C1=O]

¹H NMR (DMSO-d₆, 400 MHz): δ 7.68-7.62 (m, 1H), 7.61-7.59 (m, 2H), 7.54 (d, 1H, J=1.0 Hz), 7.49-7.47 (m, 1H), 6.84 (s, 1H), 5.06 (q, 1H, J=5.2 Hz), 4.54 (q, 2H, J=19.6 Hz), 3.28-3.12 (m, 2H). ¹³C NMR (DMSO-d₆, 100 MHz): δ172.5, 168.3, 142.6, 135.3, 134.5, 132.2, 132.0, 128.3, 123.9, 123.3, 116.3, 54.6, 47.6, 27.8. HRMS: [M−H]⁻ m/z calcd for C₁₄H₁₂N₃O₃⁻, 270.0884; found, 270.0883.

### 3-(4-Hydroxyphenyl)-2-(1-oxo-1,3-dihydro-2H-benzo[f]isoindol-2-yl)propanoic acid

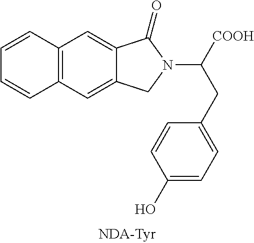 [SMILES: O=C(O)C(Cc1ccc(O)cc1)N1Cc2cc3ccccc3cc2C1=O]

¹H NMR (DMSO-d₆, 400 MHz): δ 9.46 (s, 1H), 8.23 (s, 1H), 8.06 (d, 1H, J=8.2 Hz), 8.02 (s, 1H), 8.00 (d, 1H, J=8.1 Hz), 7.63-7.53 (m, 2H), 7.05 (d, 2H, J=8.4 Hz), 6.58 (d, 2H, J=8.4 Hz), 5.11 (q, 1H, J=5.4 Hz), 4.56 (dd, 2H, J₁=17.2 Hz, J₂=3.8 Hz), 3.30/3.10 (q, 2H, J₁=6.4 Hz, J₂=8.7 Hz). ¹³C NMR (DMSO-d₆, 100 MHz): δ 171.9, 167.4, 155.8, 136.6, 134.6, 132.3, 129.9, 129.5, 129.3, 127.9, 127.8, 127.3, 126.2, 123.0, 121.9, 115.2, 55.2, 50.0, 33.8. HRMS: [M−H]⁻ m/z calcd for C₂₁H₁₆NO₄⁻, 346.1085; found, 346.1083.

### 2-(1-Oxo-1,3-dihydro-2H-benzo[f]isoindol-2-yl)-3-phenylpropanoic acid

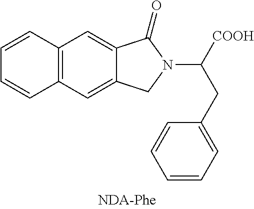 [SMILES: O=C(O)C(Cc1ccccc1)N1Cc2cc3ccccc3cc2C1=O]

¹H NMR (DMSO-d₆, 400 MHz): δ 13.2 (s, 1H), 8.27 (s, 1H), 8.11 (d, 1H, J=8.1 Hz), 8.05 (s, 1H), 8.01 (d, 1H, J=8.1 Hz), 7.64-7.55 (m, 2H), 7.28 (d, 2H, J=7.2 Hz), 7.21 (t, 2H, J=7.5 Hz), 7.12 (t, 1H, J=7.2 Hz), 5.21 (q, 1H, J=5.4 Hz), 4.60 (q, 2H, J=14.6 Hz), 3.45-3.16 (m, 2H). ¹³C NMR (DMSO-d₆, 100 MHz): δ 172.3, 167.9, 137.9, 137.0, 135.1, 132.7, 130.2, 129.8, 129.0, 128.8, 128.4, 128.1, 126.9, 126.7, 123.6, 122.5, 55.5, 47.8, 35.0. HRMS: [M+H]⁺ m/z calcd for C₂₁H₁₇NO₃H⁺, 332.1281; found, 332.1279.

### 2-(1-Oxo-1,3-dihydro-2H-benzo[f]isoindol-2-yl)propanoic acid

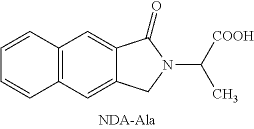 [SMILES: CC(C(=O)O)N1Cc2cc3ccccc3cc2C1=O]

¹H NMR (DMSO-d₆, 400 MHz): δ 8.36 (s, 1H), 8.16 (d, 1H, J=7.9 Hz), 8.11 (s, 1H), 8.05 (d, 1H, J=8.1 Hz), 7.73-7.56 (m, 2H), 4.92 (q, 1H, J=7.5 Hz), 4.66 (dd, 2H, J₁=17.3 Hz, J₂=4.3 Hz), 1.55 (d, 3H, J=7.5 Hz). ¹³C NMR (DMSO-d₆, 100 MHz): δ 173.0, 167.4, 136.9, 134.8, 132.4, 130.3, 129.4, 128.1, 127.8, 126.3, 123.1, 122.1, 49.4, 46.6, 15.3. HRMS: [M−H]⁻ m/z calcd for C₁₅H₁₂NO₃⁻, 254.0823; found, 254.0825.

### 2-(1-Oxo-1,3-dihydro-2H-benzo[f]isoindol-2-yl)succinic acid

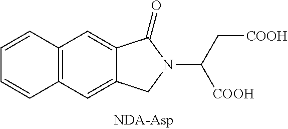 [SMILES: O=C(O)CC(C(=O)O)N1Cc2cc3ccccc3cc2C1=O]

¹H NMR (DMSO-d₆, 400 MHz): δ 12.51 (s, 1H), 8.36 (s, 1H), 8.14 (d, 1H, J=8.0 Hz), 8.10 (s, 1H), 8.03 (d, 1H, J=8.1 Hz), 7.65-7.56 (m, 2H), 5.16 (q, 1H, J=4.7 Hz), 4.60 (dd, 2H, J₁=17.1 Hz, J₂=1.6 Hz), 3.04/2.93 (q, 2H, J=7.6 Hz, J=8.6 Hz). ¹³C NMR (DMSO-d₆, 100 MHz): δ 171.8, 171.3, 167.3, 136.8, 134.8, 132.4, 129.9, 129.4, 128.0, 127.8, 126.3, 123.2, 122.1, 51.2, 47.7, 34.5. HRMS: [M−H]⁻ m/z calcd for C₁₆H₁₂NO₅⁻, 298.0721; found, 298.0717.

### 3-(1H-imidazol-4-yl)-2-(1-oxo-1,3-dihydro-2H-benzo[f]isoindol-2-yl)propanoic acid

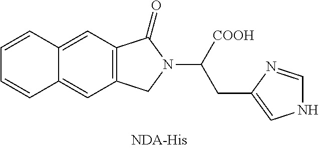 [SMILES: O=C(O)C(Cc1c[nH]cn1)N1Cc2cc3ccccc3cc2C1=O]

¹H NMR (DMSO-d₆, 400 MHz): δ 14.33 (s, 1H), 8.99 (s, 1H), 8.31 (s, 1H), 8.13 (d, 1H, J=8.2 Hz), 8.11 (s, 1H), 8.04 (d, 1H, J=8.2 Hz), 7.66-7.57 (m, 2H), 7.42 (s, 1H), 5.32 (q, 1H, J=5.2 Hz), 4.81/4.81 (d, 2H, J=16.6 Hz), 3.50-3.40 (m, 2H). ¹³C NMR (DMSO-d₆, 100 MHz): δ 170.7, 167.7, 136.6, 134.8, 133.8, 132.3, 129.5, 129.4, 129.3, 128.0, 127.9, 126.3, 123.3, 122.2, 116.8, 53.5, 46.9, 24.3. HRMS: [M−H]⁻ m/z calcd for C₁₈H₁₅N₃O₃⁻, 320.1041; found, 320.1038.

¹H and ¹³C NMR Spectra

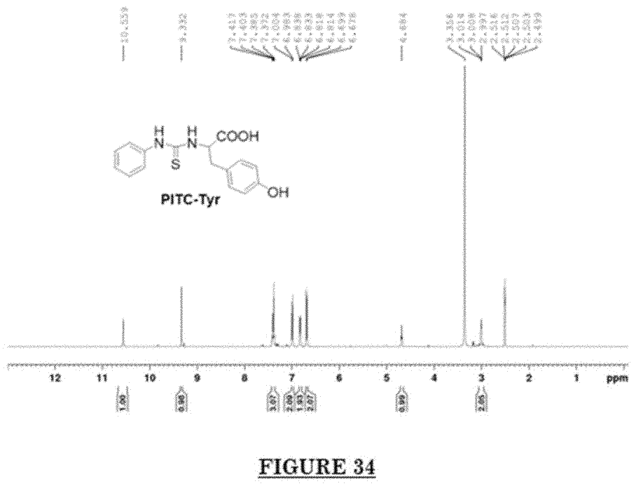

FIG. 34 shows ¹H NMR spectrum of PITC-Tyr.

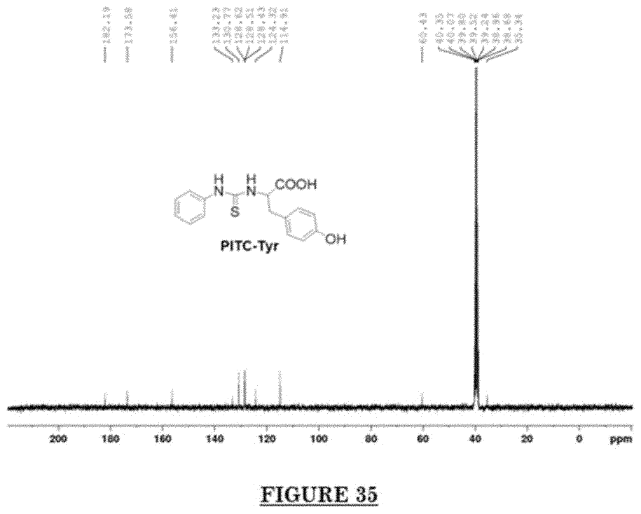

FIG. 35 shows ¹³C NMR spectrum of PITC-Tyr.

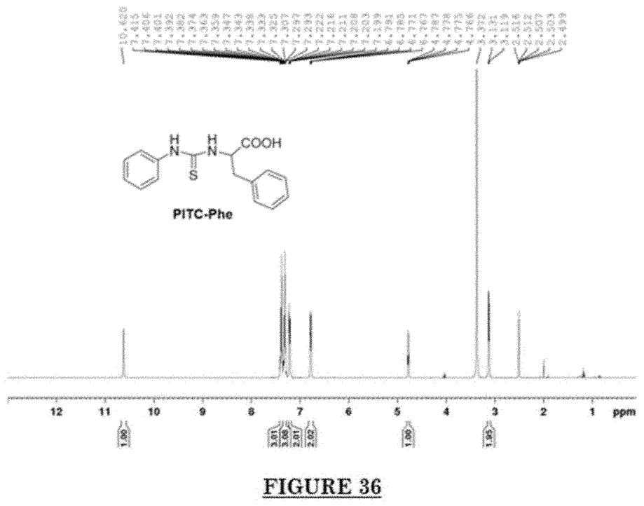

FIG. 36 shows ¹H NMR spectrum of PITC-Phe.

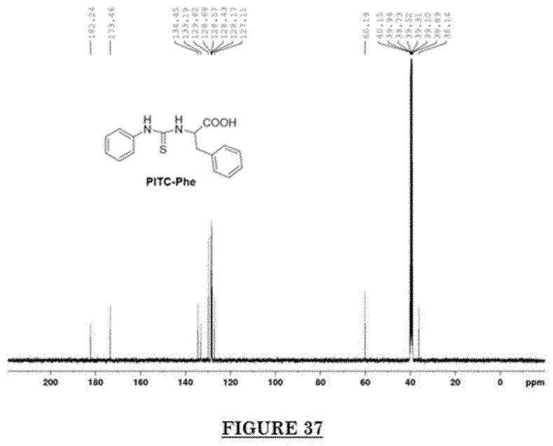

FIG. 37 shows ¹³C NMR spectrum of PITC-Phe.

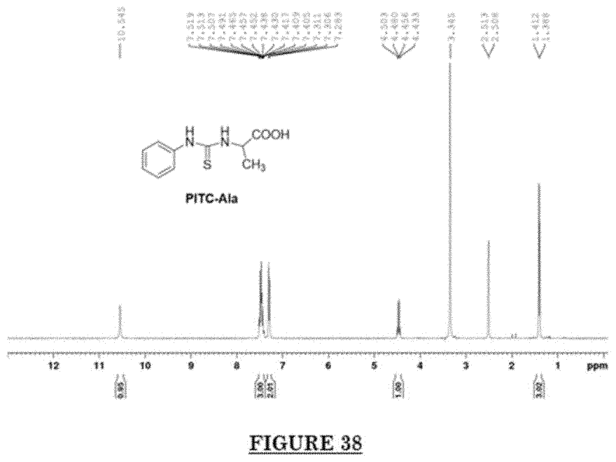

FIG. 38 shows ¹H NMR spectrum of PITC-Ala.

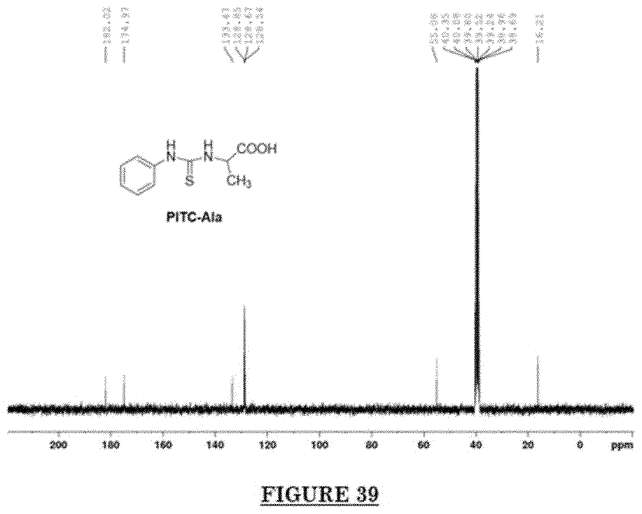

FIG. 39 shows ¹³C NMR spectrum of PITC-Ala.

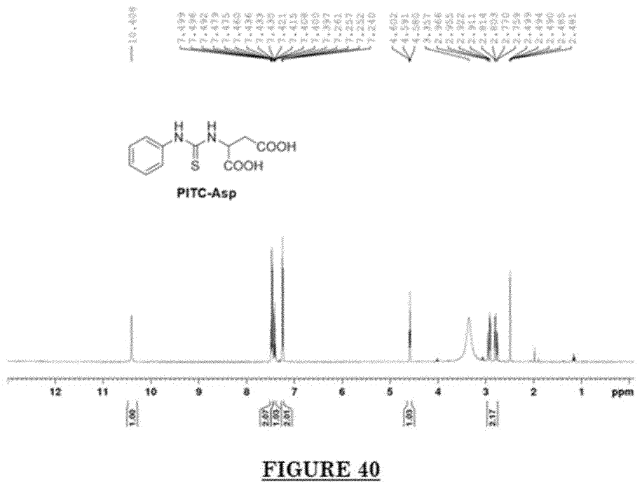

FIG. 40 shows ¹H NMR spectrum of PITC-Asp.

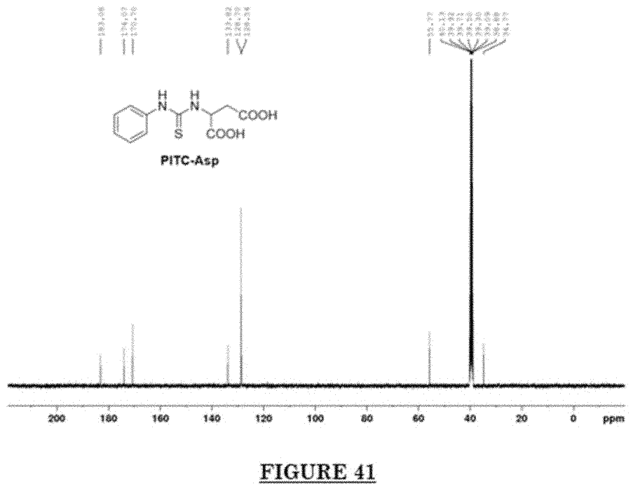

FIG. 41 shows ¹³C NMR spectrum of PITC-Asp.

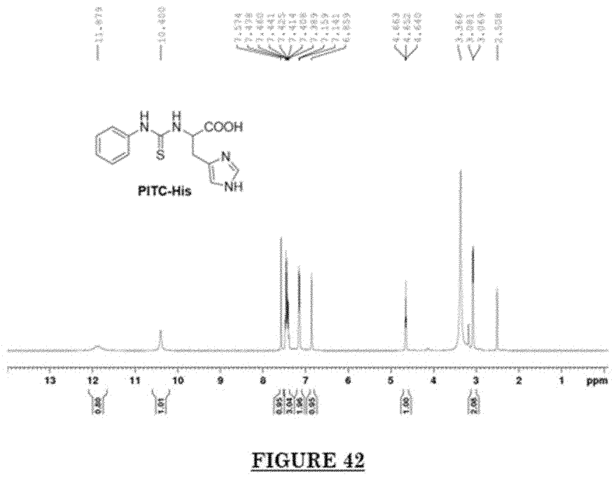

FIG. 42 shows ¹H NMR spectrum of PITC-His.

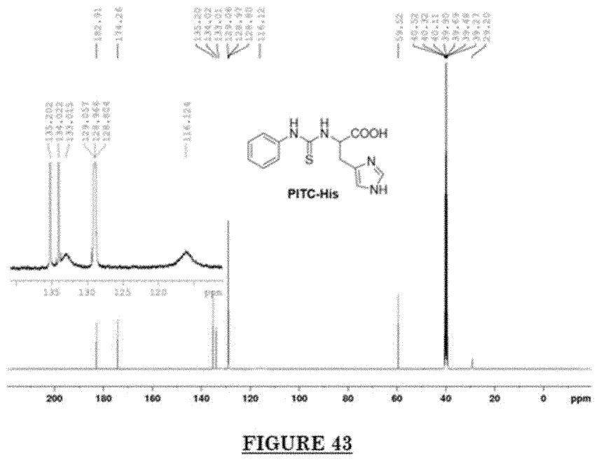

FIG. 43 shows ¹³C NMR spectrum of PITC-His.

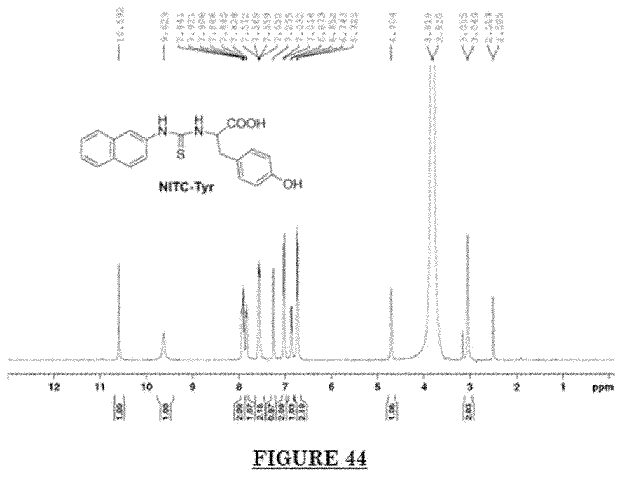

FIG. 44 shows ¹H NMR spectrum of NITC-Tyr.

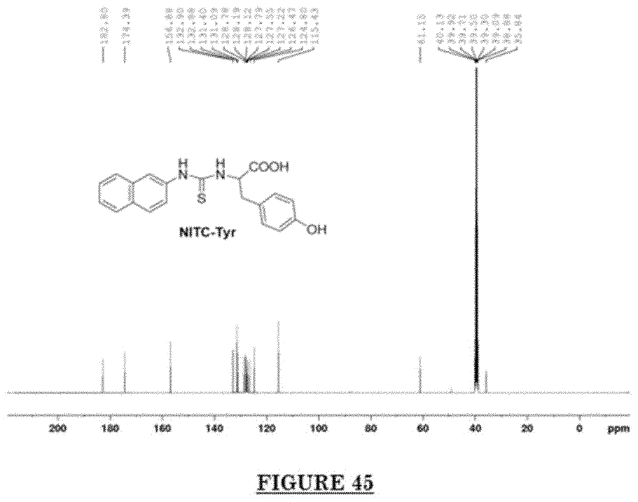

FIG. 45 shows ¹³C NMR spectrum of NITC-Tyr.

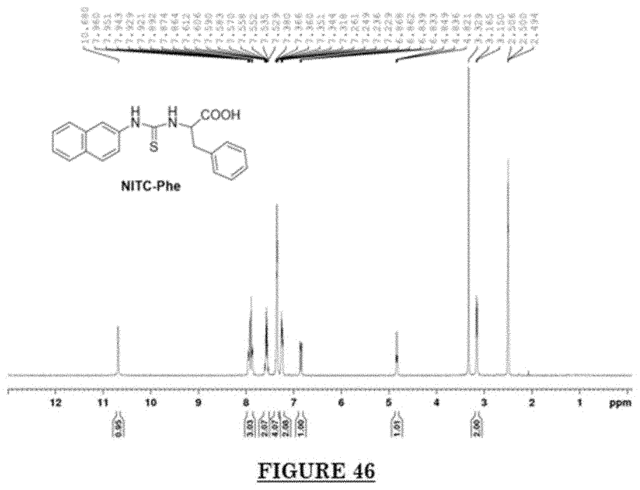

FIG. 46 shows ¹H NMR spectrum of NITC-Phe.

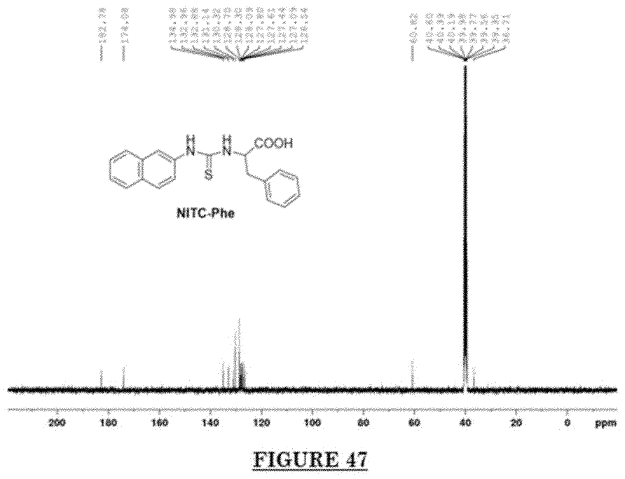

FIG. 47 shows ¹³C NMR spectrum of NITC-Phe.

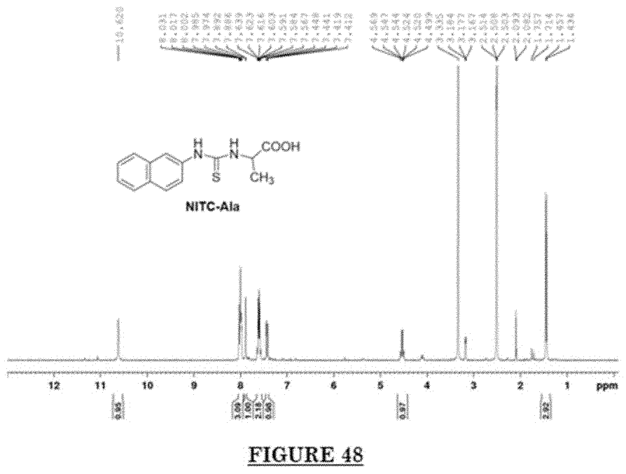

FIG. 48 shows ¹H NMR spectrum of NITC-Ala.

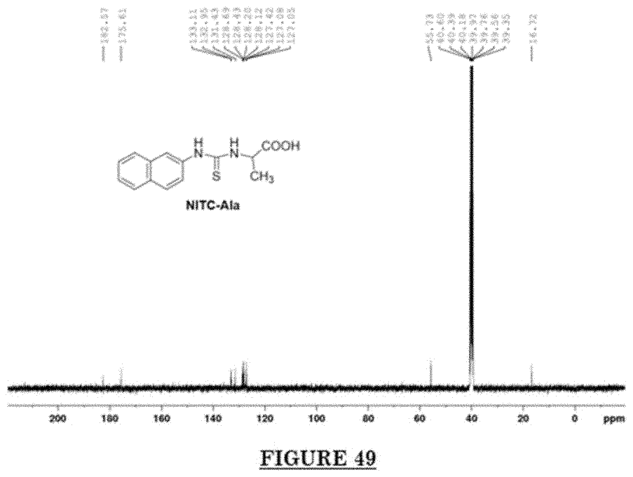

FIG. 49 shows ¹³C NMR spectrum of NITC-Ala.

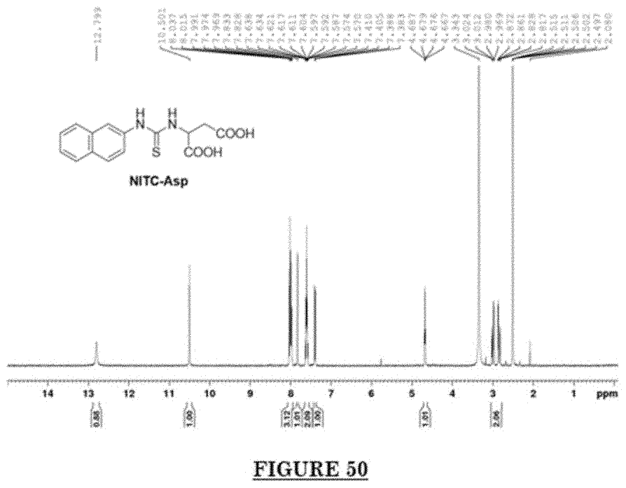

FIG. 50 shows ¹H NMR spectrum of NITC-Asp.

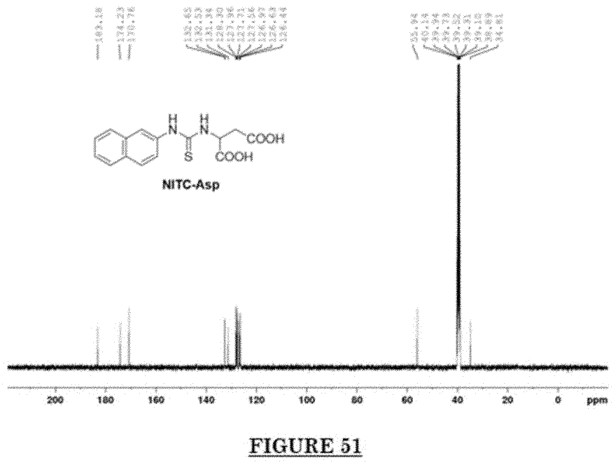

FIG. 51 shows ¹³C NMR spectrum of NITC-Asp.

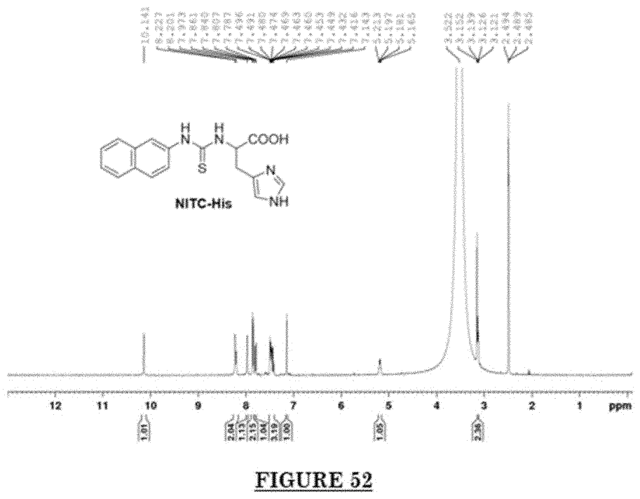

FIG. 52 shows ¹H NMR spectrum of NITC-His.

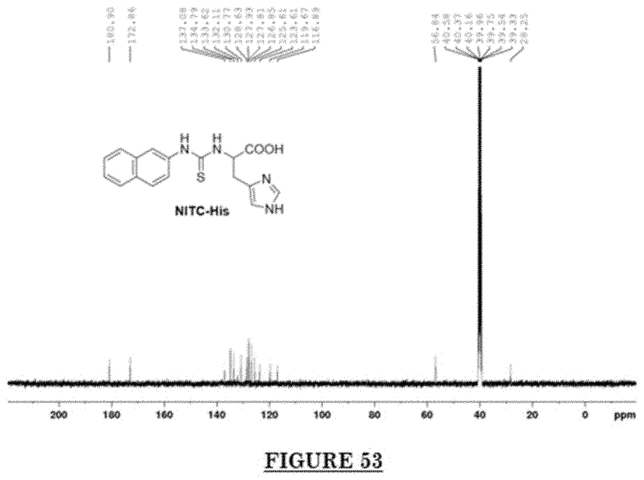

FIG. 53 shows ¹³C NMR spectrum of NITC-His.

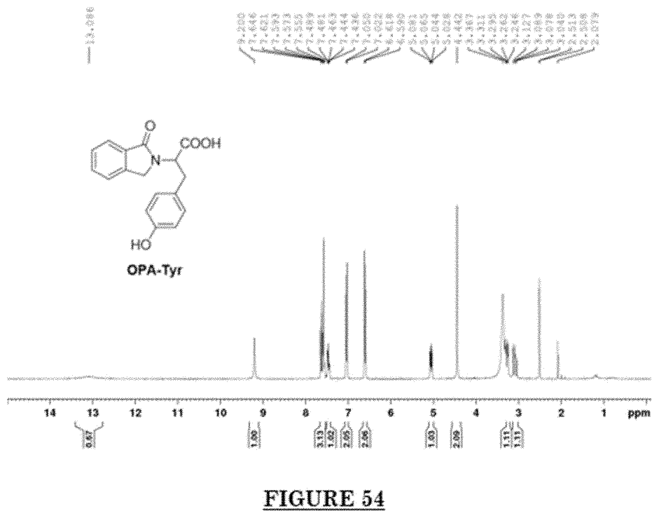

FIG. 54 shows ¹H NMR spectrum of OPA-Tyr.

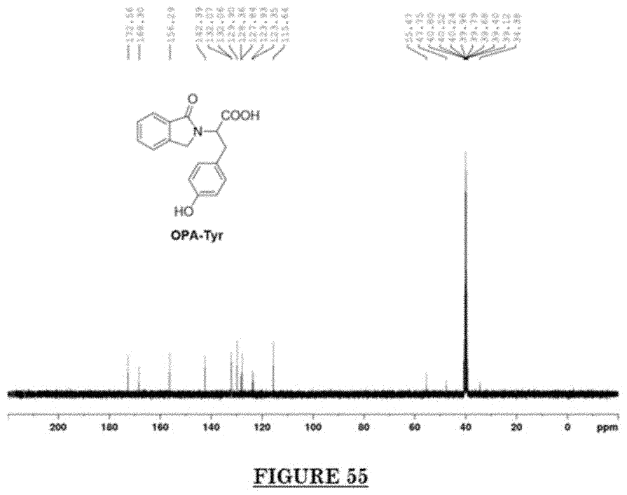

FIG. 55 shows ¹³C NMR spectrum of OPA-Tyr.

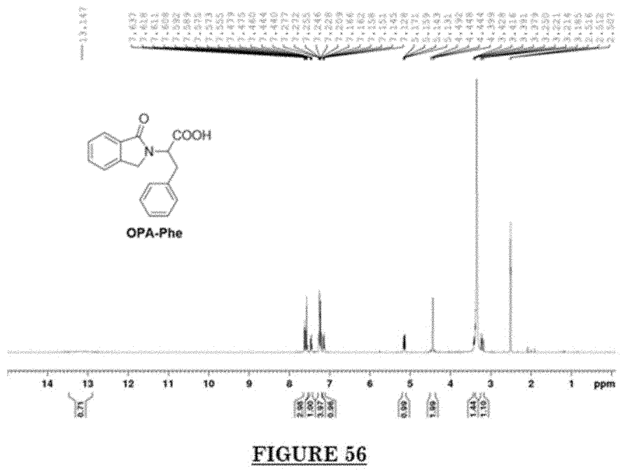

FIG. 56 shows ¹H NMR spectrum of OPA-Phe.

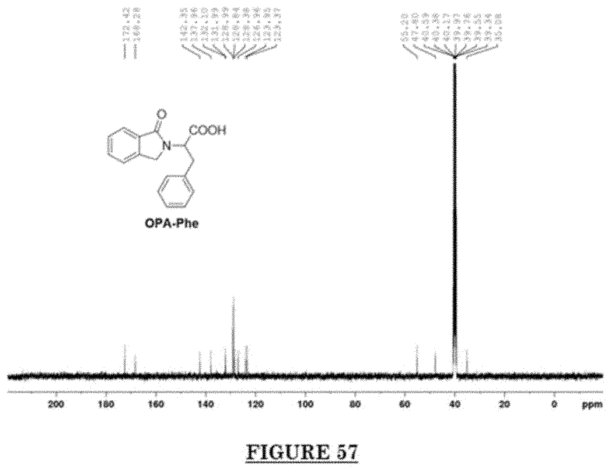

FIG. 57 shows ¹³C NMR spectrum of OPA-Phe.

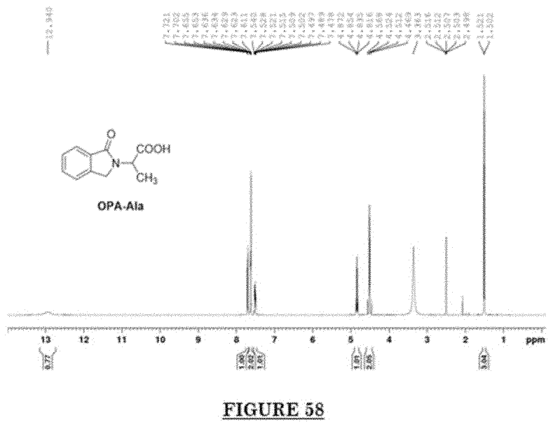

FIG. 58 shows ¹H NMR spectrum of OPA-Ala.

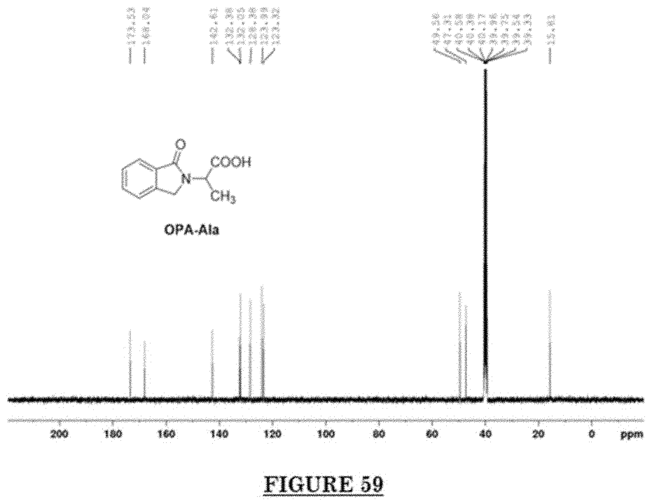

FIG. 59 shows ¹³C NMR spectrum of OPA-Ala.

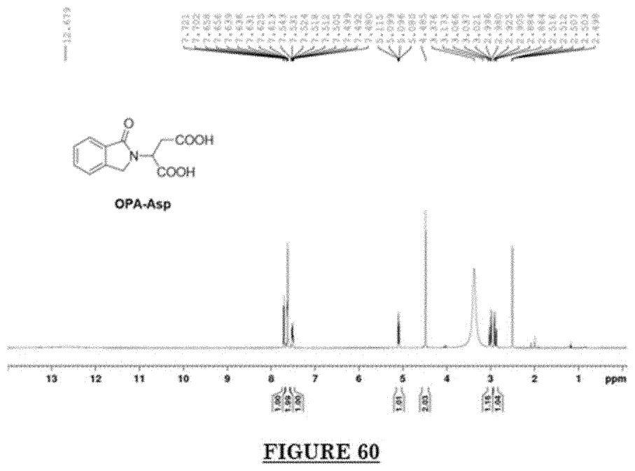

FIG. 60 shows ¹H NMR spectrum of OPA-Asp.

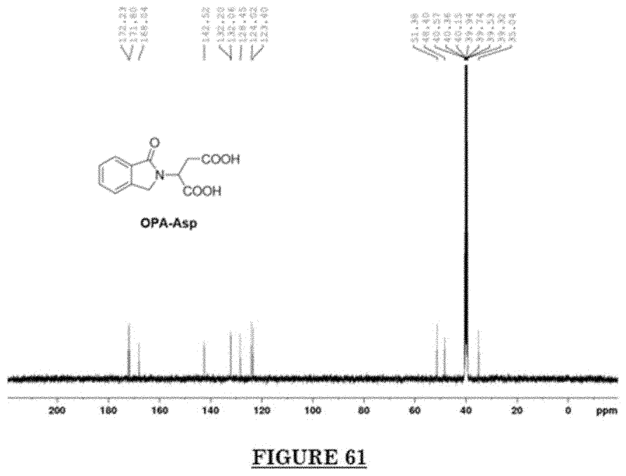

FIG. 61 shows ¹³C NMR spectrum of OPA-Asp.

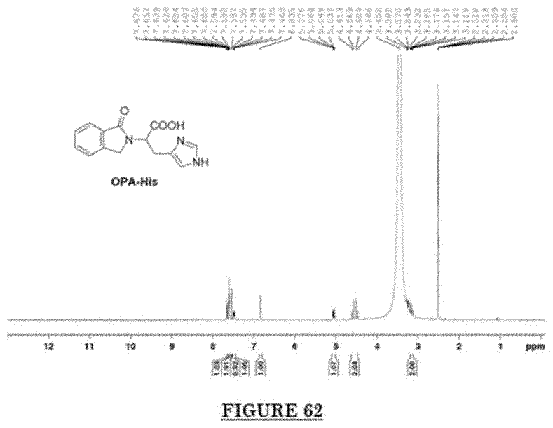

FIG. 62 shows ¹H NMR spectrum of OPA-His.

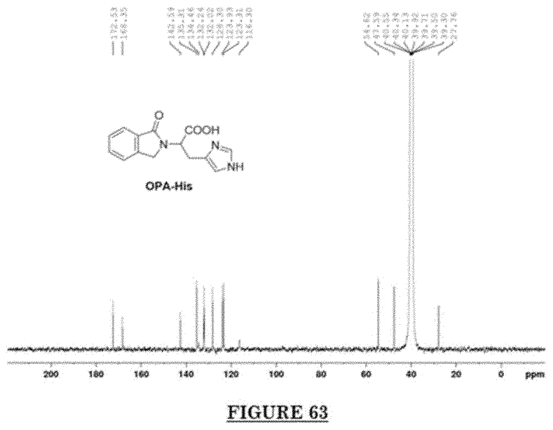

FIG. 63 shows ¹³C NMR spectrum of OPA-His.

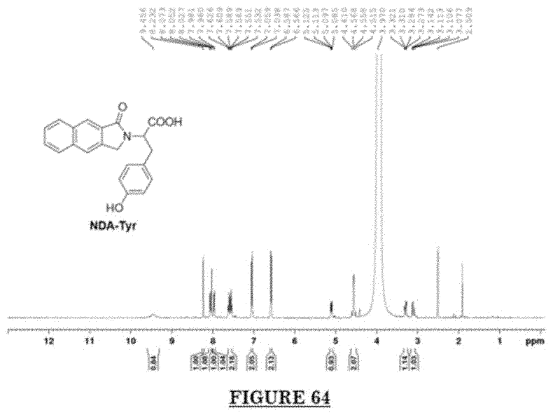

FIG. 64 shows ¹H NMR spectrum of NDA-Tyr.

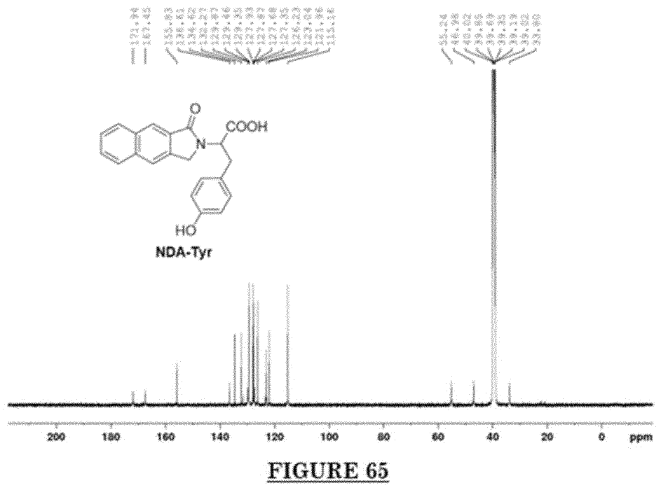

FIG. 65 shows ¹³C NMR spectrum of NDA-Tyr.

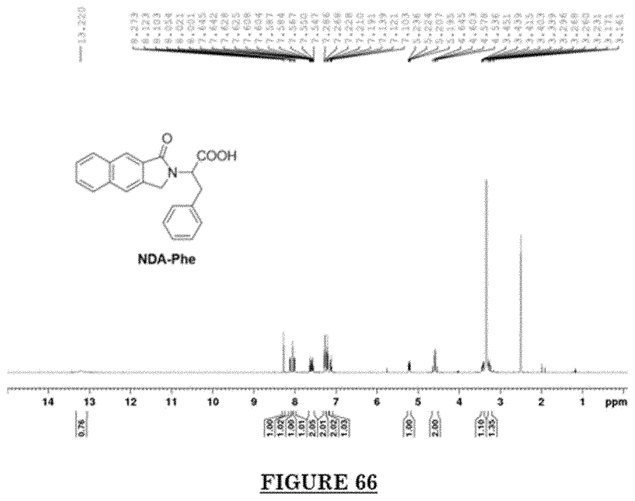

FIG. 66 shows ¹H NMR spectrum of NDA-Phe.

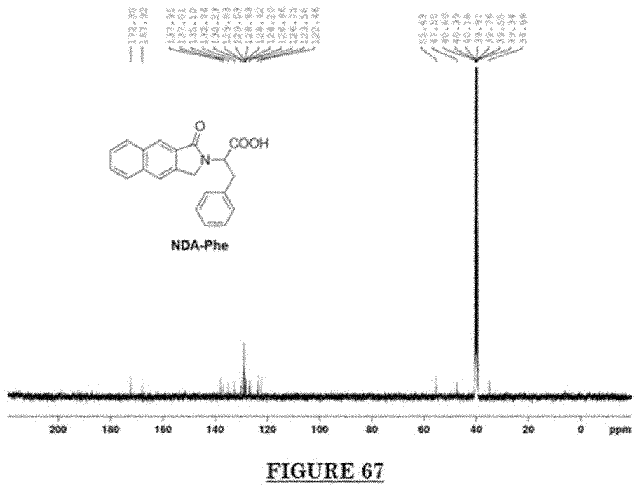

FIG. 67 shows ¹³C NMR spectrum of NDA-Phe.

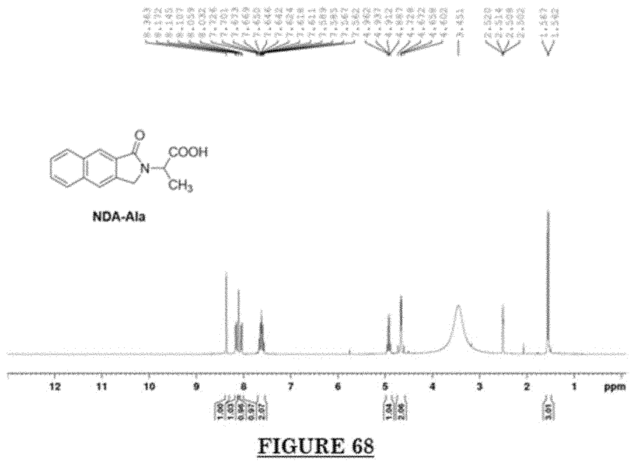

FIG. 68 shows ¹H NMR spectrum of NDA-Ala.

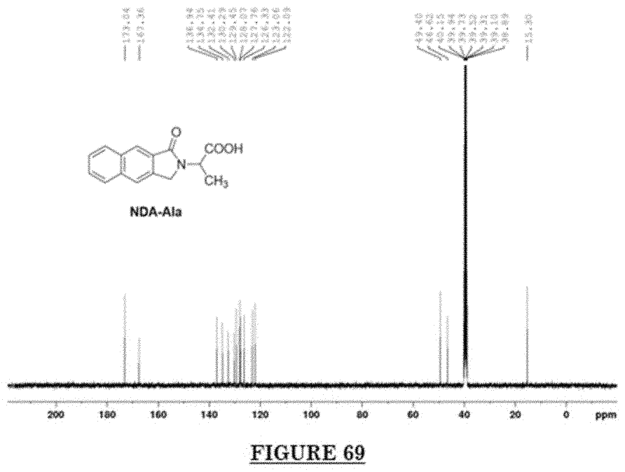

FIG. 69 shows ¹³C NMR spectrum of NDA-Ala.

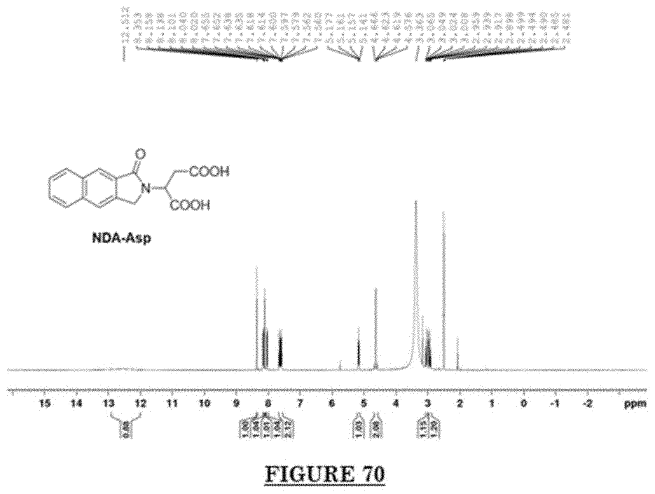

FIG. 70 shows ¹H NMR spectrum of NDA-Asp.

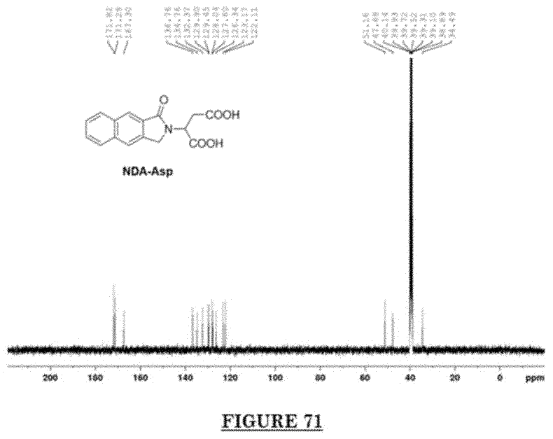

FIG. 71 shows ¹³C NMR spectrum of NDA-Asp.

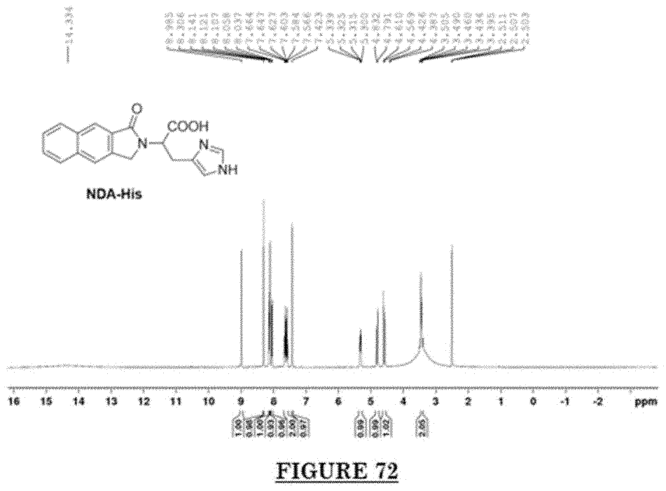

FIG. 72 shows ¹H NMR spectrum of NDA-His.

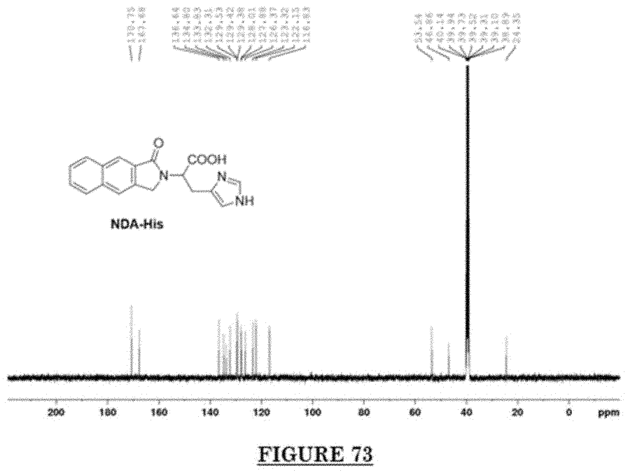

FIG. 73 shows ¹³C NMR spectrum of NDA-His.

HRMS Spectra

Samples were analyzed by infusion using a 50/50 mixture of MS grade acetonitrile/water with 0.1% formic acid in the mobile phase. The injection volume was 10 μL. The ion source was a heated electrospray source (H-ESI type II) performing in positive or negative mode. HRMS full scans were acquired from m/z 100-800 Da. The automatic gain control and mass resolution were set at 1×106 ions and 70,000 (m/z=200), respectively.

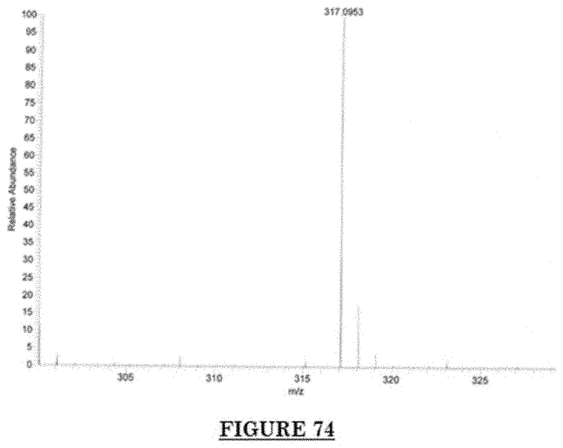

FIG. 74 shows MS spectrum of PITC-Tyr.

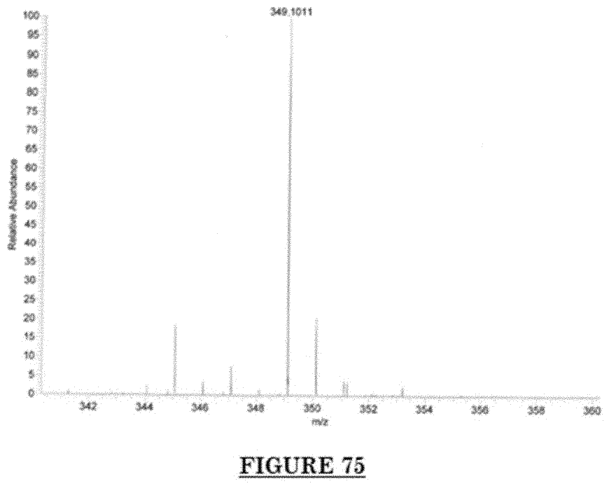

FIG. 75 shows MS spectrum of PITC-Phe.

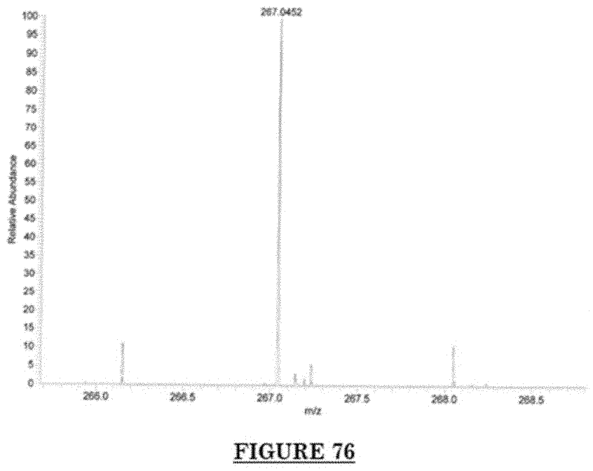

FIG. 76 shows MS spectrum of PITC-Asp.

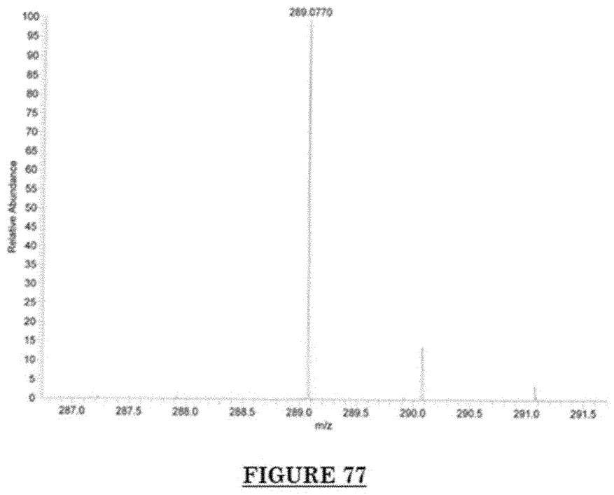

FIG. 77 shows MS spectrum of PITC-His.

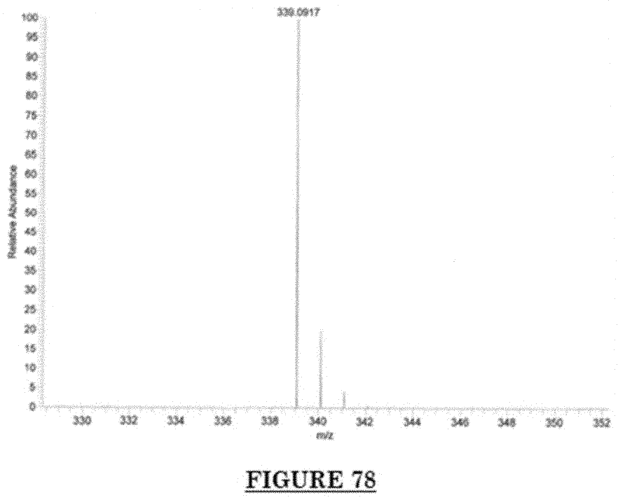

FIG. 78 shows MS spectrum of NITC-His.

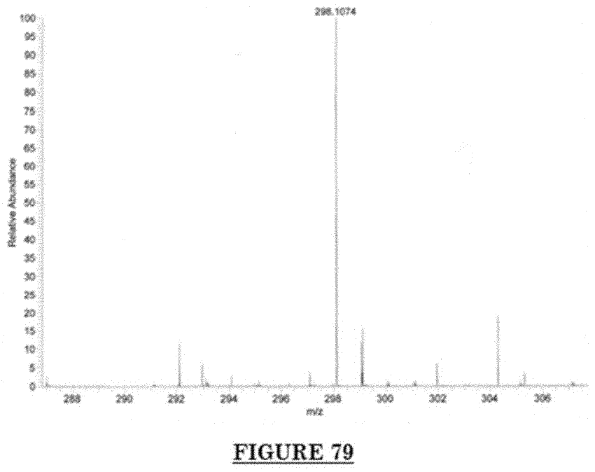

FIG. 79 shows MS spectrum of OPA-Tyr.

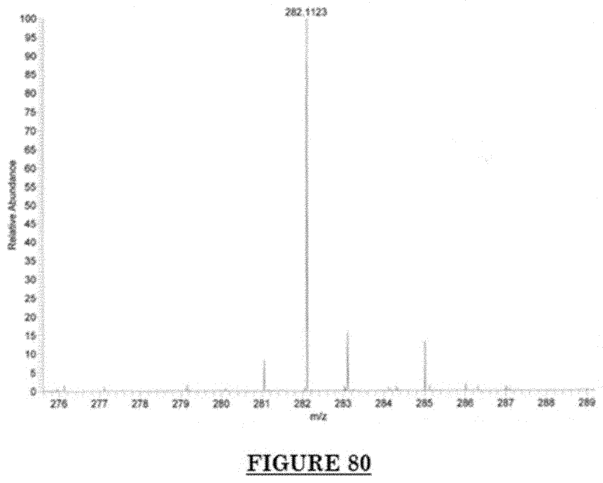

FIG. 80 shows MS spectrum of OPA-Phe.

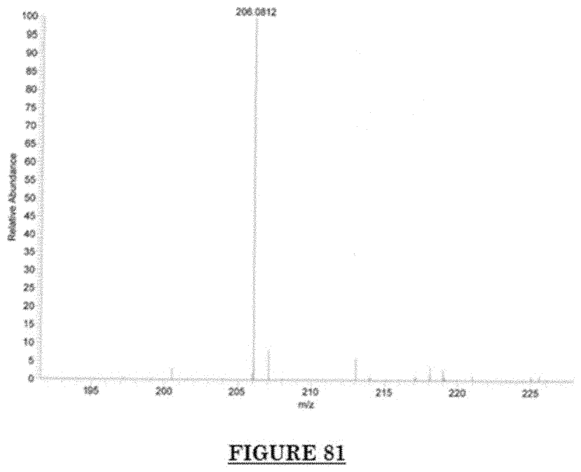

FIG. 81 shows MS spectrum of OPA-Ala.

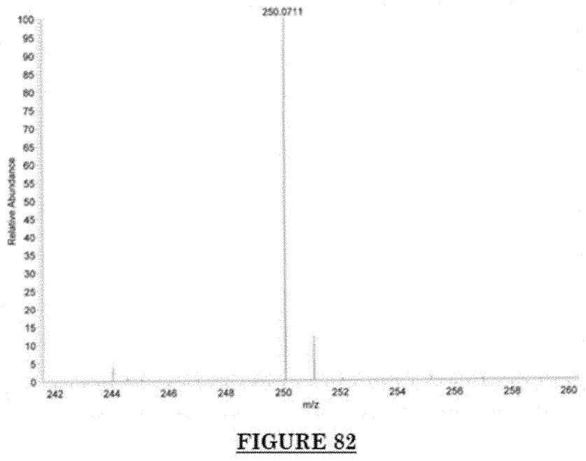

FIG. 82 shows MS spectrum of OPA-Asp.

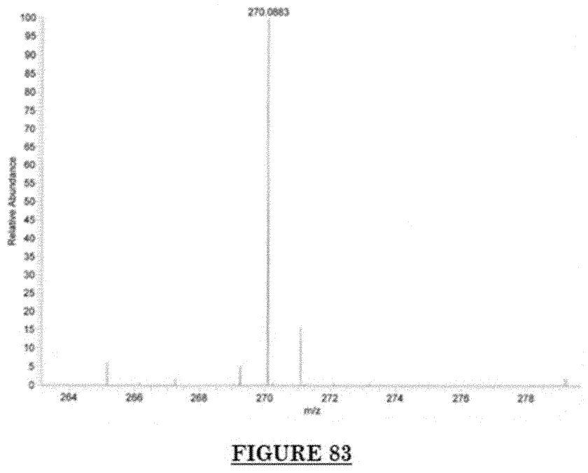

FIG. 83 shows MS spectrum of OPA-His.

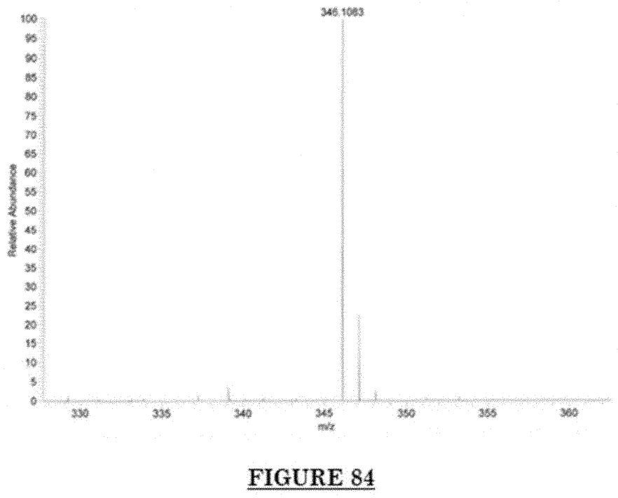

FIG. 84 shows MS spectrum of NDA-Tyr.

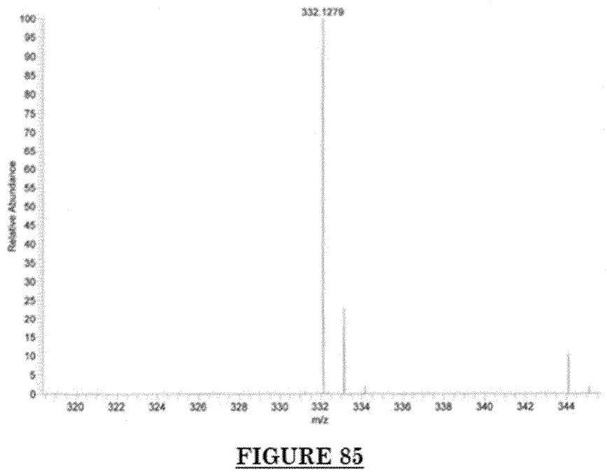

FIG. 85 shows MS spectrum of NDA-Phe.

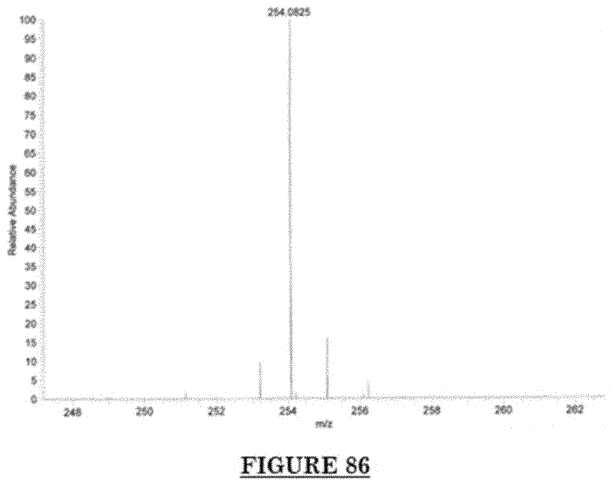

FIG. 86 shows MS spectrum of NDA-Ala.

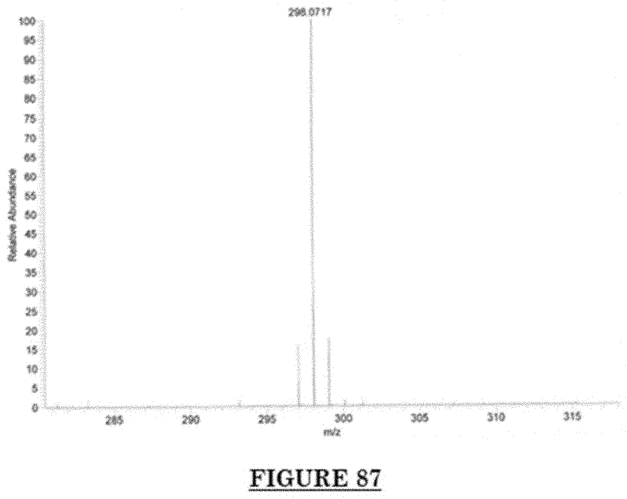

FIG. 87 shows MS spectrum of NDA-Asp.

FIG. 88 shows MS spectrum of NDA-His.

Identification of amino acids (AA) with nanopore technology remains a great challenge due to the low signal-to-noise ratio and unpredictable conformational changes of AAs during their translocation through nanopores. Here, we showed that the combination of an N-terminal derivatization strategy of AAs with nanopore technology could lead to effective in situ differentiation of AAs by testing four series of derivatives of five selected AAs, i.e. Ala, Asp, Phe, Tyr, and His using an α-Hemolysin nanopore. Results demonstrated the feasibility of derivatization-assisted identification of AAs regardless of their charge composition and polarity. The method was further applied to discriminate each individual AA in testing datasets using their established nanopore profiles from training datasets. This will not only pave a way for identification of individual AAs but also lead to future applications in protein/peptide sequencing using the nanopore technology.

In a further aspect, the current disclosure provides nanopore technology for sensing individual amino acids by a derivatization strategy and can provide methods for combining nanopore biosensing and N-terminal derivatization of amino acids to effectively differentiate individual amino acids with similar properties for potential future applications in protein sequencing.

Nanopore technology holds remarkable promise for sequencing proteins and peptides. To achieve this, it is necessary to establish a characteristic profile for each individual amino acid through the statistical description of its translocation process. However, the subtle molecular differences among all twenty amino acids along with their unpredictable conformational changes at the nanopore sensing region result in very low distinguishability. Here, the current disclosure provides the electrical sensing of individual amino acids using an α-hemolysin nanopore based on a derivatization strategy. Using derivatized amino acids as detection surrogates not only prolongs their interactions with the sensing region, but also improves their conformational variation.

Furthermore, we show that distinct characteristics including current blockades and dwell times can be observed among all three classes of amino acids after 2,3-naphthalenedicarboxaldehyde (NDA)- and 2-naphthylisothiocyanate (NITC)-derivatization, respectively. These observable characteristics were applied towards the identification and differentiation of 9 of the 20 natural amino acids using their NITC derivatives. The method demonstrated herein will pave the way for the identification of all amino acids and further protein and peptide sequencing.

The primary structure of proteins and peptides plays a significant role in their structural folding and functions. Very often subtle changes in the primary sequence of a protein can lead to debilitating pathologies. Traditional methods for proteome analysis and sequencing, such as mass spectrometry and Edman degradation, suffer from high cost, short reads, long turnaround time, and lack of sensitivity; so alternative approaches are sought. Nanopores made of either biological or inorganic materials with orifices of nanometer diameters and depth have been exploited as an exceptionally sensitive tool for the analysis of individual biomolecules in real time without the potential bias associated with signal amplification. See, J. Im, S. Lindsay, X. Wang and P. Zhang, ACS Nano, 2019, 13, 6308-6318 and M. Waugh, K. Briggs, D. Gunn, M. Gibeault, S. King, Q. Ingram, A. M. Jimenez, S. Berryman, D. Lomovtsev, L. Andrzejewski and V. Tabard-Cossa, Nat. Protoc., 2020, 15, 122-143. As a result, significant progress towards DNA and RNA sequencing has been realized through nanopore technology. Various other applications for nanopores in single molecular sensing have also been demonstrated. See, J. Nivala, D. B. Marks and M. Akeson, Nat. Biotechnol., 2013, 31, 247-250, S. Benner, R. J. A. Chen, N. A. Wilson, R. Abu-Shumays, N. Hurt, K. R. Lieberman, D. W. Deamer, W. B. Dunbar and M. Akeson, Nat. Nanotechnol., 2007, 2, 718-724, and E. V. B. Wallace, D. Stoddart, A. J. Heron, E. Mikhailova, G. Maglia, T. J. Donohoe and H. Bayley, Chem. Commun., 2010, 46, 8195-8197.

Recently, the focus of efforts has been directed towards amino acid identification and sequencing of proteins and peptides, which holds great promise for the advancement of proteomics. Research into protein and peptide nanopore applications has been reported using the ionic current blockade signatures generated by their nanopore translocation. See, G. Huang, A. Voet and G. Maglia, Nat. Commun., 2019, 10, 835. Various nanopore methods including ionic current blockade measurement in biological nanopores (i.e. bacteriophage T7 DNA packaging motor, Z. Ji, X. Kang, S. Wang and P. Guo, Biomaterials, 2018, 182, 227-233, FraC nanopores, see G. Huang, K. Willems, M. Soskine, C. Wloka and G. Maglia, Nat. Commun., 2017, 8, 935, L. Restrepo-Perez, G. Huang, P. R. Bohlander, N. Worp, R. Eelkema, G. Maglia, C. Joo and C. Dekker, ACS Nano, 2019, 13, 13668-13676, aerolysin, see L. Restrepo-Perez, G. Huang, P. R. Bohlander, N. Worp, R. Eelkema, G. Maglia, C. Joo and C. Dekker, ACS Nano, 2019, 13, 13668-13676, F. Piguet, H. Ouldali, M. Pastoriza-Gallego, P. Manivet, J. Pelta and A. Oukhaled, Nat. Commun., 2018, 9, 966., and C. Cao, N. Cirauqui, M. J. Marcaida, E. Buglakova, A. Duperrex, A. Radenovic and M. Dal Peraro, Nat. Commun., 2019, 10, 4918, and α-hemolysin, see, G. Di Muccio, A. E. Rossini, D. Di Marino, G. Zollo and M. Chinappi, Sci. Rep., 2019, 9, 6440, and inorganic perpendicular nanochannels, see P. Boynton and M. Di Ventra, Sci. Rep., 2016, 6, 25232, and recognition by tunneling current, Y. A. Zhao, B. Ashcroft, P. M. Zhang, H. Liu, S. M. Sen, W. Song, J. Im, B. Gyarfas, S. Manna, S. Biswas, C. Borges and S. Lindsay, Nat. Nanotechnol., 2014, 9, 466-473, have been used to identify proteins and peptides. Nonetheless, nanopore sequencing of proteins and peptides still faces formidable open challenges, especially the feasibility of distinguishing individual amino acids. Recently, a small group of researchers have focused on nanopore sensing of individual amino acids. See, A. J. Boersma and H. Bayley, Angew. Chem. Int. Ed., 2012, 51, 9606-9609. Y. Guo, A. Niu, F. Jian, Y. Wang, F. Yao, Y. Wei, L. Tian and X. Kang, Analyst, 2017, 142, 1048-1053, and A. Asandei, A. E. Rossini, M. Chinappi, Y. Park and T. Luchian, Langmuir, 2017, 33, 14451-14459. For example, a fingerprinting scheme has been reported in which only a subset of amino acids was labeled and detected. See, L. Restrepo-Perez, G. Huang, P. R. Bohlander, N. Worp, R. Eelkema, G. Maglia, C. Joo and C. Dekker, ACS Nano, 2019, 13, 13668-13676. In another study, an elegant approach has been developed to detect 13 of 20 proteinogenic amino acids in an aerolysin nanopore with the help of a short peptide tag. See, H. Ouldali, K. Sarthak, T. Ensslen, F. Piguet, P. Manivet, J. Pelta, J. C. Behrends, A. Aksimentiev and A. Oukhaled, Nat. Biotechnol., 2020, 38, 176-181. However, all the reported methods are unfeasible for the derivatization and differentiation of amino acids in situ.

Based on the Edman peptide degradation reaction, see P. Edman, Acta Chem. Scand., 1950, 4, 283-293, P. Edman, Arch. Biochem., 1949, 22, 475-476, I. Molnar-Perl, in Quantitation of Amino Acids and Amines by Chromatography: Methods and Protocols, ed. I. MolnarPerl, 2005, vol. 70, pp. 163-198, and R. Checa-Moreno, E. Manzano, G. Miron and L. F. Capitan-Vallvey, J. Sep. Sci., 2008, 31, 3817-3828, herein we demonstrate that the efficient N-terminal derivatization of amino acids using aromatic tags can augment the distinguishability of different amino acids when they translocate through α-hemolysin (α-HL) nanopores. A panel of nine amino acids, including nonpolar, polar and charged ones, could be discriminated individually.

This method can potentially be employed in the development of nanopore sequencing of protein or peptide analytes. The derivatization reagents, 2,3-naphthalenedicarboxaldehyde (NDA) and 2-naphthylisothiocyanate (NITC) were chosen due to their wide usage along with their high reaction rate and efficiency with most natural amino acids. See, I. Molnar-Perl, in Quantitation of Amino Acids and Amines by Chromatography: Methods and Protocols, ed. I. MolnarPerl, 2005, vol. 70, pp. 163-198, R. Checa-Moreno, E. Manzano, G. Miron and L. F. Capitan-Vallvey, J. Sep. Sci., 2008, 31, 3817-3828, M. Fountoulakis and H. W. Lahm, J. Chromatogr. A, 1998, 826, 109-134, and K. L. Woo and Y. K. Ahan, J. Chromatogr. A, 1996, 740, 41-50.

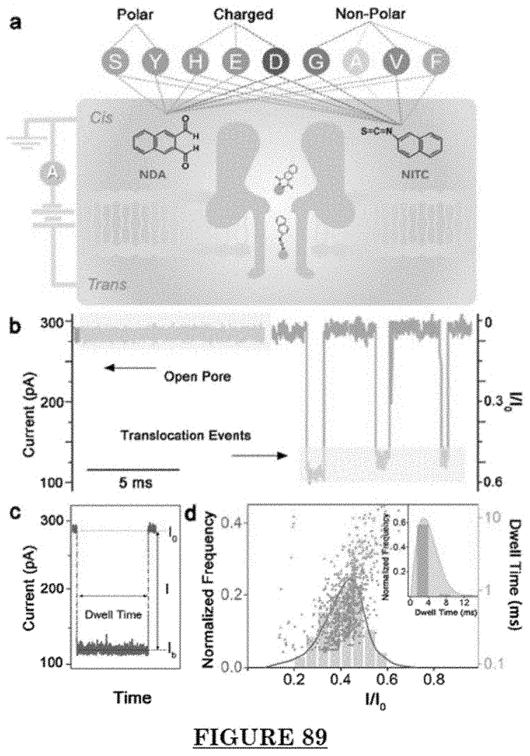

Nine amino acids were randomly selected from each of the three classes for derivatization with NDA and NITC, respectively, see FIG. 89 at a, including polar uncharged amino acids: serine (Ser, S), tyrosine (Tyr, Y); charged amino acids: histidine (His, H), glutamic Acid (Glu, E), and aspartic Acid (Asp, D); and non-polar amino acids: glycine (Gly, G), alanine (Ala, A), valine (Val, V), and phenylalanine (Phe, F). FIG. 89 shows at (a) schematic of NDA and NITC derivatization of 9 amino acids in 3 classes and the experimental setup (not to scale). An external voltage is applied to the trans side of the lipid bilayer, while the cis side is grounded. FIG. 89 at (b) shows representative fragment of ionic current recording before (open pore) and after (translocation events) NITC-Tyr derivatives were added. FIG. 89 at (c) shows illustration of typical signal events caused by translocation blockade. FIG. 89 at (d) shows histogram of events per bin of current blockade I/I₀ (left versus bottom axes) and scatter-plot of dwell time versus I/I₀ (right versus bottom axes) produced by NITC-Tyr in an α-HL nanopore. The Inset depicts the histogram of events per bin of dwell time for NITC-Tyr. The derivatization process is simple, efficient, and was performed under mild basic buffer conditions as described in synthetic routes I and II for NDA and NITC derivatives, respectively. In our nanopore analysis experiments, only purified amino acid derivatives were employed. The molecular structures of all the derivatization products were characterized and confirmed by ¹H and ¹³C nuclear magnetic resonance analyses. However, due to the high efficiency of both chemistries used in our design, they can be readily employed for N-terminal modification of amino acids in aqueous solution for practical applications.

In a typical experiment, a single α-HL nanopore is inserted into a phosphate lipid bilayer that separates cis and trans compartments in an electrolyte solution. An external positive voltage (100 mV) is applied to the trans side of the bilayer, while the cis side is electrically grounded, see FIG. 89 at a). The direction of protein insertion can be determined by the absolute value of open pore current under positive and negative voltages. See, X. Wei, Z. Zhang, X. Wang, B. Lenhart, R. Gambarini, J. Gray and C. Liu, Nanotechnol. Precis. Eng., 2020, 3, 2-8. In the present study, the tail of the mushroom shaped α-HL is inserted into the lipid bilayer, and all the samples enter the nanochannel through the head of a-HL remaining on the cis side. In the absence of amino acid derivatives, a steady ionic current I0 (B287 pA) flows through the nanopore, open pore, see FIG. 89 at (b). Each unmodified amino acid and each derivatization reagent were first tested using an α-HL nanopore, and no obvious characteristic signal was observed for an extended recording time. In contrast, addition of any amino acid derivatives on the cis side of the bilayer induces transient events of the ionic current, with a representative translocation blockade profile for NITC-Tyr shown in FIG. 89 at b.

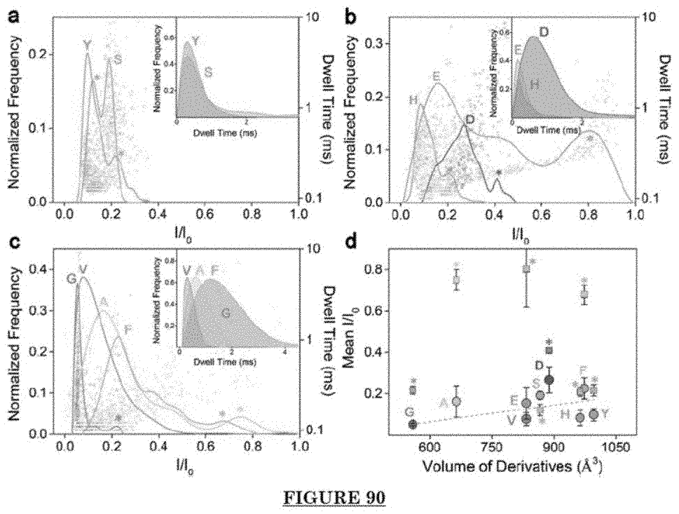

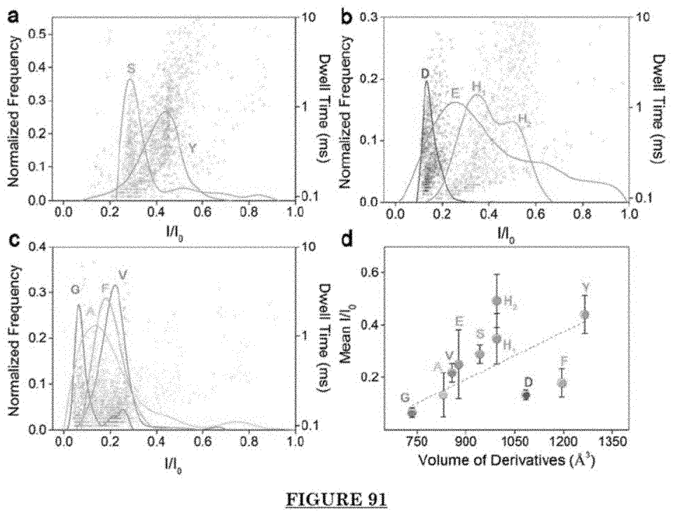

Each blockade corresponding to the capture of an individual derivative in the pore is characterized by two parameters: current blockade I/I₀(I=I₀−I_{b}, I_{b} indicates the residual current induced by the analyte) and the blockade duration (dwell time) that represents the effective interaction time between the pore and the analyte, see FIG. 89 at c. A typical histogram of the current blockades (I/I₀) or the dwell time has a single narrow peak, FIG. 89 at d, which is characterized by the mean value of a Gaussian fit and its standard deviation (SD). In the case of the NITC-Tyr in FIG. 89 at b, I/I₀ was 0.439±0.073 and the dwell time was 2.866±0.540 ms. Similar experiments were performed separately for two series of NDA and NITC derivatives of 9 different amino acids. Corresponding superimposed histograms of the relative current blockade values are summarized in FIGS. 90 and 91, where results are classified into polar uncharged, electrically charged, and non-polar uncharged amino acids. FIG. 90 shows superimposed histograms of I/I₀ obtained from nanopores for NDA-modified amino acids, analyzed individually and grouped as FIG. 90 at: (a) polar, (b) charged, and (c) non-polar amino acids. Insets in a-c are superimposed histograms of dwell time for the derivatives. FIG. 90 at (d) shows mean relative current blockade and standard deviation produced by each NDA amino acid derivative versus its spatial volume. The grey dashed line is obtained by a linear fit of the main peaks (represented by circles). All data were acquired in 3 M KCl, 10 mM Tris-HCl buffer, at 8.0 pH, 200 mM derivative concentration, and under a 100 mV bias applied to the trans side. For each histogram, at least 600-1000 events were analyzed. Overlaps of current blockade subpopulations between each other are tagged with asterisks. FIG. 91 shows superimposed histograms of I/I₀ obtained from the nanopore for NITC-modified amino acids, analyzed individually and grouped as FIG. 91 at: (a) polar, (b) charged, and (c) non-polar amino acids. FIG. 91 at (d) shows mean relative current blockade and standard deviation produced by each NITC amino acid derivative versus its spatial volume. The grey dashed line is obtained by a linear fit of the main peaks (represented by the circle). All data were acquired in 3 M KCl, 10 mM Tris-HCl buffer, at 8.0 pH, 200 mM derivative concentration, and under a 100 mV bias applied to the trans side. For each histogram, at least 600-1000 events were analyzed.

For the identification of individual amino acids, current blockade (I/I0) is used as the primary criterion as it can reflect the variation of the spatial structure of the molecule directly before and after modification, as confirmed previously. See, F. Piguet, H. Ouldali, M. Pastoriza-Gallego, P. Manivet, J. Pelta and A. Oukhaled, Nat. Commun., 2018, 9, 966. Dwell time is used as the secondary identification criterion when the current blockade is noneffective.

FIG. 90 at a-c shows the superimposed histograms of each class of amino acids after modification with NDA, in which case three rigid benzoisoindolones are formed after linking with amino acids. This modification is expected to give a larger volume and various spatial structures to each individual amino acid, resulting in a fingerprint signal when translocated through the constriction region of the α-HL nanopore (width: 1.4 nm). The NDA derivatives all have effective interactions with the nanopore and each NDA derivative has its own characteristic distribution of I/I₀ and dwell time, see FIG. 90 at a-c. Furthermore, these characteristic signals from different amino acids in each class are clearly distinguishable.

For instance, although two populations—corresponding to members (Y, and S) of the polar family, FIG. 90 at a, have similar dwell time distributions (inset in FIG. 90 at a), they can be clearly identified and distinguished from one another with mean I/I₀ of 0.099±0.029 and 0.192±0.018, respectively.

Compared with the polar family, NDA derivatives of the charged family H, E, and D exhibit wider distributions of I/I₀, with the mean I/I₀ of 0.084±0.037, 0.154±0.077, and 0.267±0.063, respectively, as shown in FIG. 90 at b. A broad distribution of the current blockade was observed for NDA-Glu (E), possibly due to its negative charge and weak hydrophilicity, and the rigid benzoisoindolone structure resulting in multiple spatial orientations inside the nanopore. Although there is a serious current blockade overlap between E and D caused by the broad distribution, they can be further distinguished by their dwell time distributions (inset in FIG. 90 at b). In the non-polar family shown in FIG. 90 at c, G and V can be distinguished by using both I/I₀ and the dwell time distribution, while A and F can be effectively distinguished by dwell time distribution regardless of their considerably overlapped I/I₀ distribution (inset in FIG. 90 at c).

After geometrically optimizing the NDA amino acid derivatives to gain an accurate measurement of hydrodynamic volume (FIG. 90 at d), we plotted the mean relative current blockade against the hydrodynamic volume to probe the physical mechanism underlying the current blockade induced by different amino acids in the nanopore. Overall, the mean I/I₀ increased as the excluded volume increases for each amino acid derivative with a fitting slope of 2.67, which agrees with the tendency found in previous reports. See, H. Ouldali, K. Sarthak, T. Ensslen, F. Piguet, P. Manivet, J. Pelta, J. C. Behrends, A. Aksimentiev and A. Oukhaled, Nat. Biotechnol., 2020, 38, 176-181, G. Baaken, I. Halimeh, L. Bacri, J. Pelta, A. Oukhaled and J. C. Behrends, ACS Nano, 2015, 9, 6443-6449, 44 A. E. Chavis, K. T. Brady, G. A. Hatmaker, C. E. Angevine, N. Kothalawala, A. Dass, J. W. F. Robertson and J. E. Reiner, ACS Sens., 2017, 2, 1319-1328.

However, subpopulations located beside the main peaks (tagged with asterisks in FIG. 90 at d) have random and irregular mean I/I₀ distribution tendency. For most NDA derivatives, both criteria (current blockade and dwell time) are needed for differentiation among amino acids. This is likely due to the fact that the NDA modification cannot induce sufficient variation between amino acids, such as the narrow volume range, i.e., from 559 to 996 Å3. In addition, the rigid benzoisoindolone structure formed in each NDA derivative causes complexity in its spatial conformation when translocating the nanopore, leading to a wide distribution on the histograms, which further dampens the discriminatory power.

To improve the discrimination between amino acids, we further modified these 9 amino acids with NITC. An increase in spatial structure complexity of the NITC-derivatives is confirmed by the wider volume range, i.e., 734-1264 Å3, which is expected to lead to higher discriminatory power and less uncertainty of the spatial orientation of derivatives inside the nanopore.

Results of all NITC derivatives confirm effective interactions with the nanopore by characteristic distribution of I/I₀ and dwell time for each derivative. Although with some notable exceptions, the superimposed histograms of each family of derivatives exhibit well-separated populations with narrow distributions, see FIG. 91. Two populations corresponding to S and Y of the non-polar amino acid family can be clearly identified and distinguished from one another by mean I/I0 values of 0.289±0.035 and 0.439±0.073, respectively, see FIG. 91 at a. Three populations corresponding to E, D, and H of the charged amino acid family also can be clearly identified and distinguished by their characteristic distributions of I/I₀, which are centered at 0.249±0.132 (E), 0.134±0.019 (D), 0.347±0.096 (Hi), and 0.492±0.201 (112), respectively. Two equally distributed peaks observed for NITC-His (H) may be attributed to the rigidity of the molecular structure of His. The NITC modification strategy shows a more obvious advantage for identification of G, A, F, and V in the non-polar family. As shown in FIG. 91 at c, these four amino acids can be discriminated through the mean I/I₀ values: 0.065±0.019 (G), 0.132±0.085 (A), 0.178±0.055 (F), and 0.217±0.036 (V), regardless of minor overlaps between each other. NITC derivatization clearly affords a wider distribution of I/I0 (0.1-0.5) across the amino acids tested compared to a previous report, see H. Ouldali, K. Sarthak, T. Ensslen, F. Piguet, P. Manivet, J. Pelta, J. C. Behrends, A. Aksimentiev and A. Oukhaled, Nat. Biotechnol., 2020, 38, 176-181, which should improve identification sensitivity.

While identification of all the 9 amino acids can be achieved by using the mean I/I₀ (primary criterion) only, their dwell time distribution (secondary criterion) was also analyzed to further enhance the identification accuracy, see FIG. 91. Similar to the NDA derivatives, the NITC derivatives also exhibited an increasing trend of the mean relative current blockade with increased hydrodynamic volume, but with a larger fitting slope of 6.04 that indicates enhanced discriminatory power by NITC derivatization, see FIG. 91 at d.

Previous studies have demonstrated that an aerolysin nanopore with a narrower constriction of B1.0 nm is able to detect a bare cysteine, see B. Yuan, S. Li, Y. L. Ying and Y. T. Long, Analyst, 2020, 145, 1179-1183, and differentiate certain peptides with one amino acid difference in length. See, F. Piguet, H. Ouldali, M. Pastoriza-Gallego, P. Manivet, J. Pelta and A. Oukhaled, Nat. Commun., 2018, 9, 966. A recent advancement demonstrates detection of more types of amino acids using a peptide as the carrier, resulting in various I/I₀ distributions around 0.4 with only slight shifts for different modified amino acids, see H. Ouldali, K. Sarthak, T. Ensslen, F. Piguet, P. Manivet, J. Pelta, J. C. Behrends, A. Aksimentiev and A. Oukhaled, Nat. Biotechnol., 2020, 38, 176-181, whereas the NITC derivatization produced larger difference between I/I₀ distributions of different amino acids (0.1-0.5), indicating improved sensitivity using small molecules as amino acid modifiers. In addition, it is overwhelmingly challenging to apply the peptide carrier method to practical protein sequencing due to various reaction conditions for modifying different amino acids. As demonstrated in the Edman degradation, N-terminal derivatization can efficiently increase the spatial size of all amino acids with similar reactivity within the same reaction, and thus can be readily applied to recognize all the amino acids towards protein sequencing.

When an analyte translocates the nanopore under the applied voltage and the diffusion effect, a signal on the electrical current trace characterized by a current blockade and a dwell time can be generated as a result of the transient occupation of the nanopore lumen by the analyte and the interaction between the analyte and the nanopore. The low frequencies of translocation events for the selected amino acids demonstrate their weak interactions with the lumen of the α-HL nanopore, due to the smaller van der Waals radii of the amino acids (B0.3-0.4 nm) compared to the dimension of the α-HL nanopore constriction region (1.4 nm). See, J. J. Kasianowicz, E. Brandin, D. Branton and D. W. Deamer, Proc. Natl. Acad. Sci. U.S.A., 1996, 93, 13770-13773, J. Wilson, L. Sloman, Z. He and A. Aksimentiev, Adv. Funct. Mater., 2016, 26, 4830-4838, and E. Kennedy, Z. Dong, C. Tennant and G. Timp, Nat. Nanotechnol., 2016, 11, 968.

As confirmed by the molecular structure modeling results, most NITC derivatives produced current blockades that positively correlate to their spatial size. Although exceptions (i.e. D, and F) were observed, the general mechanism of electrical sensing of individual amino acids is to increase their spatial size by derivatization to promote interactions with the nanopore sensing region, thereby improving the signal-to-noise ratio. Further investigation using other types of biological nanopores is warranted to probe possible explanations for these exceptions, such as intra- and inter-molecular interactions between analytes. See, X. Y. Zhang, C. C. Gong, O. U. Akakuru, Z. Q. Su, A. G. Wu and G. Wei, Chem. Soc. Rev., 2019, 48, 5564-5595.

In conclusion, we have demonstrated a derivatization strategy for reliable identification of individual amino acids using an α-HL nanopore. Compared to bare amino acids, both NDA-derived and NITC-derived amino acids can produce obvious fingerprint signals when translocating the nanopore. Furthermore, the amino acids S, Y, D, E, H, G, A, F, and V can be effectively identified with improved discriminatory power by NITC derivatization. While promising results were obtained for 9 amino acids, we do recognize the overall complexity of identifying all 20 amino acids. In particular, we need to develop more effective conjugation chemistry to derivatize proline, which does not have a primary amino group like the others. Additionally, an in-depth analysis is needed to better understand the interactions between amino acid derivatives and the lumen surface of biological nanopores. Novel characterizations of stochastic signals other than the traditional blockade and dwell time must be explored, and more advanced data analysis technology (e.g. machine learning, pattern recognition, etc.) should be applied to achieve even greater resolution.

Nonetheless, compared to previous efforts on amino acid identification using nanopores, the presented method is readily applicable to future protein sequencing. We provide a “sequencing-by-hydrolysis” method, in which a nanopore will be used to identify the N-terminal amino acid of each peptide fragment in a peptide ladder generated from a peptide analyte, and then bioinformatics methods will be applied to reconstitute its full-length sequence. See, H. Y. Zhong, Y. Zhang, Z. H. Wen and L. Li, Nat. Biotechnol., 2004, 22, 1291-1296.

All patents, patent applications, published applications, and publications, databases, websites and other published materials referred to throughout the entire disclosure herein, unless noted otherwise, are incorporated herein by reference in their entirety.

Various modifications and variations of the described methods of the disclosure will be apparent to those skilled in the art without departing from the scope and spirit of the disclosure. Although the disclosure has been described in connection with specific embodiments, it will be understood that it is capable of further modifications and that the disclosure as claimed should not be unduly limited to such specific embodiments. Indeed, various modifications of the described modes for carrying out the disclosure that are obvious to those skilled in the art are intended to be within the scope of the disclosure. This application is intended to cover any variations, uses, or adaptations of the disclosure following, in general, the principles of the disclosure and including such departures from the present disclosure come within known customary practice within the art to which the disclosure pertains and may be applied to the essential features herein before set forth.

What is claimed is:

1. A method for identifying individual amino acids comprising:
employing a biosensing strategy using at least one nanopore;
N-terminal derivatization of at least one amino acid to form an amino acid analyte; and
differentiating individual amino acid analytes from one another via analysis of the at least one analyte interacting with the at least one nanopore.

2. The method of claim 1 further comprising developing a characteristic profile for each individual amino acid via a statistical description of each individual amino acid analyte's translocation process through the at least one nanopore.

3. The method of claim 1 further comprising analyzing blockade and dwell times for the individual amino acid analytes within the at least one nanopore.

4. The method of claim 1, wherein the nanopore is an α-hemolysin nanopore.

5. The method of claim 1, further comprising employing an aromatic tag as part of the N-terminal derivatization.

6. The method of claim 1, wherein N-terminal derivatization uses derivatization reagents comprising 2,3-naphthalenedicarboxaldehyde (NDA) and/or 2-naphthylisothiocyanate (NITC).

7. The method of claim 1, wherein identifying at least one individual amino acid is accomplished via analyzing current blockade induced via presence of the at least one amino acid analyte.

8. The method of 7, further comprising identifying at least one individual amino acid is accomplished via analyzing dwell time induced via the at least one amino acid analyte when analyzing current blockage is ineffective at identifying the at least one amino acid.

9. The method of 1, further comprising generating a signal on an electrical current trace characterized by current blockade and dwell time when the at least one individual amino acid analyte translocates the at least one nanopore.

10. A method for identifying individual amino acids comprising:
inserting at least one nanopore into a phosphate lipid bilayer wherein the phosphate lipid bilayer separates cis and trans compartments in an electrolyte solution;
applying an external positive voltage to a trans facing side of the bilayer;
grounding a cis facing side of the bilayer;
determining amino acid analyte insertion via an absolute value of open pore current under positive and negative voltages; and
identifying at least one individual amino acid via interaction of an amino acid analyte with the at least one nanopore.

11. The method of claim 10, further comprising wherein a tail of the at least one nanopore is inserted into the phosphate lipid bilayer with a head of the at least one nanopore remaining in the cis compartment.

12. The method of claim 10, wherein the at least one nanopore comprises α-hemolysin nanopore.

13. The method of claim 10, further comprising introducing a sample of at least one individual amino acid analyte to the cis compartment.

14. The method of claim 10, wherein introduction of at least one amino acid derivative in the cis compartment induces transient events in an ionic current flowing through the at least one nanopore.

15. The method of claim 10, further comprising characterizing capture of at least one amino acid analyte via analysis of current blockade and blockade duration within the at least one nanopore.

16. The method of claim 10, wherein identifying at least one individual amino acid is accomplished via analyzing current blockade induced via presence of the at least one individual amino acid analyte.

17. The method of 16, further comprising identifying at least one individual amino acid is accomplished via analyzing dwell time induced via presence of the at least one individual amino acid analyte when analyzing current blockage is ineffective at identifying the at least one amino acid analyte.

18. The method of 10, further comprising generating a signal on an electrical current trace characterized by current blockade and dwell time when the at least one individual amino acid analyte translocates the at least one nanopore.
